(19) United States
(12) Patent Application Publication
(10) Pub. No.: US 2013/0150810 A1
(43) Pub. Date: Jun. 13, 2013

(54) INTRAVAGINAL RING FOR THE DELIVERY OF UNIQUE COMBINATIONS OF ANTIMICROBIAL COMPOSITIONS

(71) Applicant: The Population Council, Inc., New York, NY (US)

(72) Inventors: Robin A. Maguire, Ossining, NY (US); Mitchell Thorn, Astoria, NY (US); David M. Phillips, Piermont, NY (US); Naomi Rutenberg, Washington, DC (US); Thomas M. Zydowsky, Brooklyn, NY (US); Shweta Ugaonkar, Elmhurst, NY (US); Samantha Seidor, New York, NY (US)

(73) Assignee: THE POPULATION COUNCIL, INC., New York, NY (US)

(21) Appl. No.: 13/759,981

(22) Filed: Feb. 5, 2013

Related U.S. Application Data
(63) Continuation-in-part of application No. 12/587,405, filed on Oct. 6, 2009, now Pat. No. 8,367,098.
Application No. 12/587,405 is a continuation-in-part of application No. 10/977,001, filed on Oct. 29, 2004, now abandoned.
Application No. 10/977,001 is a continuation of application No. PCT/US03/13456, filed on Apr. 30, 2003.
(60) Provisional application No. 60/376,400, filed on Apr. 30, 2002.
(60) Provisional application No. 60/377,050, filed on May 1, 2002.

(51) Int. Cl. A61M 31/00

(52) U.S. Cl. CPC A61M 31/00
USPC 604/285; 427/2.1

(57) ABSTRACT

Disclosed are compositions for inhibiting transmission of a sexually transmitted infection that contain one or more polyanionic microbicides, such as carrageenans, including lambda carrageenan, as well as water-soluble metal salts and specified antiretroviral agents comprising NNRTIs and NRTIs. Also disclosed are methods for making and using the compositions. Also disclosed are intravaginal rings for delivering water-soluble compounds, and preferably high molecular weight water-soluble polymers at essentially a zero order rate. The rings include an outer layer of non-water-swellable elastomer, and preferably high molecular weight water-soluble polymer and an inner layer of the water-soluble polymer, which is imbedded in the outer layer, and an aperture through the outer layer for release thereof only through that aperture.

### CROSS-REFERENCE TO RELATED APPLICATIONS

This application is a continuation-in-part of U.S. patent application No. 12/587,405, filed on Oct. 6, 2009, which is a continuation-in-part of U.S. patent application Ser. No. 10/977,001, filed on Oct. 29, 2004, abandoned, which is a continuation of International Application No. PCT/US03/13456, filed on Apr. 30, 2003, which claims the benefit of the filing date of U.S. Provisional Patent Application No. 60/376,400, filed on Apr. 30, 2002, and U.S. Provisional Patent Application No. 60/377,050, filed on May 1, 2002, the disclosures of which are incorporated herein by reference.

### BACKGROUND OF THE INVENTION

Carrageenans are polysaccharides obtained from the red algae commonly known as seaweed. They are a structural component of seaweed and are extracted as three main types, namely iota, kappa and lambda, although there are other types as well, including kappa-II, mu and nu carrageenans. Carrageenans have been used extensively in the food, pharmaceutical and cosmetics industries as thickeners, gelling agent, and stabilizing and dispersing agents. Extensive pharmacological and toxicological studies have been conducted. Carrageenan has been found to be non-toxic by oral, denial, and inhalation routes of administrations even at extremely high doses. The carrageenans were therefore classified as “generally recognized as safe” (GRAS) by the FDA in 1972². Further extensive oral pharmacokinetic studies conducted in pigs, rats, mice, gerbils, guinea pigs, ferrets, hamsters, dogs, and monkeys³⁻¹¹ showed that the breakdown of the carrageenans in the gastrointestinal tract were minimal at best and that absorption was virtually non-existent

International Patent Publication WO 94/15624 teaches use of sulfated polysaccarides such as iota carrageenan, dextran sulfate, kappa carrageenan, lambda carrageenan heparin mimetics, heparin sulfate, pentosan polysulfate, chondrotin sulfate, lentinan sulfate, curdlan sulfate, de-N-sulfated heparin and fucoidan, to inhibit cell-to-cell transmission of HIV and thus the sexual transmission of Acquired Immune Deficiency Syndrome (AIDS) as well as Chlamydia organism. This publication teaches that iota carrageenan is the most efficacious of the commercially available sulfated carrageenans in preventing HIV infection and in blocking Chlamydia infection in vitro and in vivo.

There have also been continuing efforts to deliver compounds such as the carrageenans intravaginally. International Patent. Publication No. WO 2012/024605 discloses a chamber for sustained release of water-soluble molecules and for use in an intravaginal device. However, the chamber in this disclosure, which is entirely separate from the intravaginal ring itself, includes a closed container or pod made of plastic or metal with release orifices in the bottom of the pod which contains a pellet of the water-swellable polymer. Furthermore. Baum et al., “An intravaginal ring for the simultaneous delivery of multiple drugs,” J. Pharm. Sci., 101, 8, 2833 discloses intravaginal delivery of microbicide combinations which once again include separate pods which are imbedded in the vaginal ring itself and in which multiple pods can thus release multiple drugs thereby. This author discloses forming pellets as a drug core which is then coated with a release polymer such as polylactic acid. The search has therefore continued for more efficient mechanisms for delivering these compounds, preferably at a zero order rate of release.

### SUMMARY OF THE INVENTION

Applicants have discovered that a certain carrageenan or mixtures or combinations of various carrageenans possess specific physical and chemical properties and that when they are formulated for vaginal administration, they provide a prolonged antimicrobial effect and inhibit or reduce the possibility of transmission of a sexually transmitted infection (STI). Applicants have also discovered a novel delivery system for the intravaginal delivery of these types of compounds.

Accordingly, a first aspect of the present invention is directed to an aqueous antimicrobial composition, comprising an effective amount of an antimicrobial agent comprising carrageenans (referred to herein as “the carrageenans” or a “carrageenan mixture”) which are lambda carrageenan in an amount of at least about 50% by dry weight of said carrageenans, remainder of said carrageenans being at least one non-lambda carrageenan, and a physiologically acceptable pH controlling agent. For purposes of the present invention, the term “antimicrobial” is meant to embrace anti-bacterial and/or antiviral activity.

A related aspect of the present invention is directed to a sexually transmitted infection (STI) inhibiting composition, comprising an effective amount of an antimicrobial agent comprising carrageenans which are lambda carrageenan in an amount of at least about 50% by dry weight of said carrageenans, remainder of said carrageenans being at least one non-lambda carrageenan, and a physiologically acceptable pH controlling agent.

The compositions may further include another antimicrobial agent and/or a vaginally administrable drug, in which case the carrageenan component may be a lambda carrageenan, without any non-lambda carrageenan. The additional agent may be in admixture and/or associated with the carrageenans such as in the form of a complex. Accordingly, a further aspect of the present invention is directed to aqueous antimicrobial composition, comprising: (a) a physiologically acceptable pH controlling agent; and (b) an effective amount of an antimicrobial agent comprising a complex of a lambda carrageenan or carrageenans which are lambda carrageenan in an amount of at least about 50% by dry weight of said carrageenans, remainder of said carrageenans being at least one non-lambda carrageenan, and an antimicrobial, physiologically acceptable water-soluble cationic metal salt.

A further aspect of the present invention is directed to an aqueous antimicrobial composition, comprising: (a) a physiologically acceptable pH controlling agent; (b) an effective amount of an antimicrobial agent comprising a complex of a lambda carrageenan or carrageenans which are lambda carrageenan in an amount of at least about 50% by dry weight of said carrageenans, remainder of said carrageenans being at least one non-lambda carrageenan; and (c) a liposulfonic acid.

A further aspect of the present invention is directed to an aqueous antimicrobial composition, comprising: (a) a physiologically acceptable pH controlling agent; (b) an effective amount of an antimicrobial agent comprising a complex of a lambda carrageenan or carrageenans which are lambda carrageenan in an amount of at least about 50% by dry weight of said carrageenans, remainder of said carrageenans being at least one non-lambda carrageenan; and (c) a vaginally administrable drug such as a contraceptive agent or an agent for hormone replacement therapy.

A further aspect of the present invention is directed to an aqueous antimicrobial composition comprising an effective amount of an antimicrobial agent comprising a polyanionic microbicide, such as carrageenans which are lambda carrageenans in an amount of at least about 50% by dry weight of the carrageenans, the remainder of the carrageenans being at least one non-lambda carrageenan, a physiologically acceptable water soluble cationic metal salt, and a non-nucleoside reverse transcriptase inhibitor or a nucleoside reverse transcriptase inhibitor.

A further aspect of the present invention is directed to a method of processing, refining or stabilizing the carrageenans of the present invention. The method entails mixing a lambda carrageenan or the carrageenans in anhydrous or powdery form with the dry form of the PH controlling agent., followed by hydration of the carrageenans e.g., by the addition of water or another aqueous solution. The method overcomes several disadvantages associated with current techniques for processing high concentrations of carrageenans into homogenous aqueous solutions and facilitates further processing into pharmaceutical formulations such as the aforementioned compositions and complexes.

The present invention is also directed to a vaginal ring for the delivery of water-soluble compounds comprising an outer layer of a non-water-swellable elastomer, an inner layer of the water-soluble compound, and at least one aperture in the outer layer to permit release of the water-soluble compound from the inner layer solely through the at least one aperture. Preferably, the water-soluble polymer comprises a high molecular weight water-soluble polymer. Preferably, the outer layer comprises an extruded polymeric, ring, and the inner layer is encased within the extruded polymeric ring whereby the outer layer comprises the only layer encasing the inner layer. In a preferred embodiment, the non-water-swellable elastomer is capable of controllably diffusing a water-insoluble compound therethrough. In one embodiment, the high molecular weight water-soluble polymer is free of any polymeric coating or layer which would prevent the release of the high molecular weight water-soluble polymer therefrom. In another embodiment, the inner layer is encased in a separate sheath, which may or may not include an active agent, for separating the inner layer from other components awl/or active agents within the IVR.

In accordance with one embodiment of the vaginal ring of the present invention, the extruded polymeric ring comprises a thermosetting or thermoplastic polymer such as EVA or silicone polymers.

In accordance with another embodiment of the vaginal ring of the present invention, the outer layer comprises an extruded polymeric ring and the vaginal ring includes a polymeric sheath surrounding the inner layer such that the at least one aperture is provided in both the outer layer and the polymeric, sheath. This embodiment is particularly advantageous when used in connection with a variety of active agents or other compounds which are incompatible, and/or which need to be maintained separately within the IVR itself. Thus, for example, in this embodiment the polymeric sheath can include an active agent such as the water soluble or water insoluble compounds discussed in more detail below. The extruded polymeric ring itself can include at least one water insoluble compound which is preferably compounded with the non-water-swellable elastomer which constitutes the ring itself. Indeed, the outer layer can include a plurality of these water insoluble compounds. In a preferred embodiment, these water insoluble compounds can comprise and NNRTI, such as MIV-150, either alone or combined with other water insoluble compounds, such as in a preferred embodiment, contraceptives such as levonorgestrel and the like. Furthermore, these embodiments include an inner layer which, aside from the water soluble compounds hereof, can also include one or more of these water insoluble compounds.

In accordance with this embodiment of the vaginal ring in the present invention, the polymeric sheath can preferably comprise a non-water-swellable elastomer, and most preferably the same as the elastomer of the non-water-swellable elastomer of the outer layer, which can be free of any additional components such as water insoluble compounds, or contain such active agents including such water insoluble compounds.

In accordance with one embodiment of the vaginal ring of the present invention, the inner layer comprises a compressed core of the high molecular weight water-soluble polymer. In a preferred embodiment, the high molecular weight water-soluble polymer comprises carrageenan. In another embodiment, the outer layer includes at least one water-insoluble compound. Preferably, this water-insoluble compound is compounded with the non-water-swellable elastomer. In a highly preferred embodiment, the at least one water-insoluble compound comprises MIV-150.

In another embodiment of the vaginal ring of the present invention, the inner layer includes a low molecular weight water-soluble compound, such as a zinc salt. In another embodiment, the inner layer comprises a pair of inner layers, preferably a first inner layer comprising a compressed core of the high molecular weight water-soluble polymer and a second inner layer comprising a core of the low molecular weight water-soluble compound. Preferably, the second inner layer includes an elastomeric polymer which enables the sustained release of the low molecular weight water-soluble compound therefrom.

In accordance with another embodiment of the vaginal ring of the present invention, the outer layer is in the form of a ring, the ring including the at least one aperture extending substantially transversely through at least a portion of the ring, and wherein the inner layer comprises a compressed pellet of the high molecular weight water-soluble polymer disposed in the at least one aperture. Preferably, the ring includes a plurality of the apertures, and the inner layer comprises a plurality of the compressed pellets of the high molecular weight water-soluble polymer disposed in the plurality of apertures.

In another embodiment, however, the ring includes a plurality of the apertures, and the inner layer comprises at least one compressed pellet of the high molecular weight water-soluble polymer disposed in at least one of the plurality of apertures, and at least one pellet of a low molecular weight water-soluble compound disposed in at least one other of the plurality of apertures. In a preferred embodiment, the at least one pellet of the low molecular weight water-soluble compound includes an elastomeric polymer which enables sustained release of the low molecular weight water-soluble compound therefrom.

In another embodiment of the vaginal ring of the present invention, the ring includes at least one low molecular weight water-insoluble compound, most preferably MIV-150.

In accordance with another embodiment of the vaginal ring of the present invention, the outer layer comprises at least one ring of the non-water-swellable elastomer, and the inner layer comprises a compressed core of the high molecular weight water-soluble polymer contained within the center of the ring. In a preferred embodiment, the ring includes an upper surface and a lower surface, and the outer surface includes a pair of outer sheets of non-water-swellable elastomer disposed on the upper and lower surfaces of the ring, the at least one aperture extending through at least one of the pair of outer sheets. Preferably, the non-water-swellable elastomer of the ring and the non-water-swellable elastomer of the pair of sheets comprises the same non-water-swellable elastomer. Preferably these water-impermeable polymers comprise EVA or a silicone polymer.

In accordance with another embodiment of the vaginal ring of the present invention, the outer layer comprises a plurality of rings of the non-water-swellable elastomer and the inner layer comprises a plurality of compressed cores of the high molecular weight water-soluble polymer contained within the centers of each of the plurality of rings. In a preferred embodiment., the outer layer comprises a plurality of rings of the non-water-swellable elastomer and the inner layer comprises a plurality of compressed cores of the high molecular weight water-soluble polymer contained within the centers of each of the plurality of rings.

In accordance with one embodiment of the vaginal ring of the present invention, the outer layer comprises a plurality of rings of the non-water-swellable elastomer and the inner layer comprises at least one inner layer comprising a compressed core of the high molecular weight water-soluble polymer disposed in at least one of the plurality of rings and at least one other inner layer of a core of a low molecular weight water-soluble compound disposed in at least one other of the plurality of rings. In a preferred embodiment, the core of low molecular weight water-soluble compound includes an elastomeric polymer which enables the sustained release of the low molecular weight water-soluble compound therefrom.

In a preferred embodiment, the vaginal ring of the present invention includes a separate ring encompassing the at least one ring. Preferably, the separate ring comprises a polymer such as a thermoplastic or thermosetting elastomer and includes a low molecular weight water-insoluble compound therein. Preferably, the low molecular weight water-insoluble compound is compounded with the polymer comprising the separate ring.

A further aspect of the present invention is directed to a method of preparing a vaginal ring for delivery of a water-soluble compound. Preferably, this aspect of the present invention comprises preparing an outer layer of a non-water-swellable elastomer, preparing an inner layer of the water-soluble compound, disposing the inner layer within the outer layer, and providing at least one aperture in the outer layer whereby the water-soluble compound can only be delivered through the at least one aperture. In a preferred embodiment, the water-soluble compound comprises a high molecular weight water-soluble polymer. Preferably, preparing the outer layer comprises forming the outer layer in the form of a ring. In another embodiment, preparing the inner layer of the high molecular weight water-soluble polymer comprises leaving the outer surface of the inner layer free of any polymeric coating or layer which would prevent the release of the high molecular weight water-soluble polymer therefrom. In another embodiment, however, the inner layer can be contained within a polymeric sheath to separate any other active agents within the sheath from active agents otherwise within the IVR.

In accordance with another embodiment of the method of the present invention, preparing the inner layer of the water soluble polymer comprises disposing the inner layer within a polymeric sheath. The polymeric sheath preferably comprises either a water-swellable or non-water-swellable polymer, preferably a non-water-swellable polymer and most preferably the same non-water-swellable polymer that comprises the outer layer constituting the ring. In accordance with this embodiment, the polymeric sheath can also include an active agent, such as a water insoluble compound. The providing of at least one aperture in the outer layer thus includes providing the at least one aperture also in the polymeric sheath, again so that the water soluble compound in the inner layer can be delivered through the at least one aperture, in this case through these two layers.

In accordance with another embodiment of the method of the present invention, preparing the outer layer comprises forming a portion of the ring including an inner surface and an outer surface and preparing a second portion of the ring including an inner surface and an outer surface, and combining the first and second portions of the ring by juxtaposing the inner surfaces of the first and second portions to fully form the ring. In a preferred embodiment, the method includes providing at least one groove in the inner surface of one of the first and second portions of the ring. In a preferred embodiment, the method includes disposing the high molecular weight water-soluble polymer in the at least one groove prior to combining the first and second portions of the ring. In accordance with another embodiment, however, the method includes providing at least one groove on the inner surfaces of each of the first and second portions of the ring. Preferably, this method includes disposing the high molecular weight water-soluble polymer in one of the at least two grooves and disposing a low molecular weight water-soluble compound in the other of the at least two grooves. In this embodiment, the low molecular weight water-soluble polymer preferably includes an elastomer, either hydrophilic or hydrophobic, which provides for the sustained release of the low molecular weight water-soluble compound therefrom.

In accordance with another embodiment of the method of the present invention, the method includes providing the at least one aperture transversely through the ring. Preferably, the at least one aperture substantially traverses the depth of the ring. In a preferred embodiment, disposing the inner layer within the outer layer comprises inserting a compressed pellet of the inner layer within the at least one aperture.

In accordance with another embodiment of the method of the present invention, the method includes providing a plurality of the apertures transversely through the ring. Preferably, this plurality of apertures substantially transverses the depth of the ring. In a preferred embodiment, disposing of the inner layer within the outer layer comprises inserting the high molecular weight water-soluble polymer within the plurality of apertures. In one aspect of this method of the present invention, the method includes combining the non-water-swellable elastomer with a low molecular weight water-insoluble compound.

In accordance with one embodiment of the method of the present invention, disposing the inner layer within the outer layer comprises inserting the high molecular weight water-soluble polymer in at least one of the plurality of apertures, and includes inserting a low molecular weight water-soluble compound in at least one other of the plurality of apertures. Preferably, the high molecular weight water-soluble polymer comprises carrageenan and the low molecular weight water-soluble compound comprises a zinc salt.

In accordance with another embodiment of the method of the present invention, disposing the inner layer within the outer layer comprises providing the compressed high molecular weight water-soluble polymer within the center of the ring. Preferably, the ring includes an upper surface and a lower surface, and the providing of the outer layer comprises providing a first sheet of non-water-swellable elastomer on the upper surface of the ring and providing a second sheet of non-water-swellable elastomer on the lower surface of the ring, and wherein the at least one aperture is disposed in one of the first and second sheets. In a preferred embodiment, preparation of the outer layer comprises forming a plurality of outer layers in the form of a plurality of rings. Preferably, preparation of the inner layer comprises compressing a plurality of cores of the high molecular weight water-soluble polymer. In a preferred embodiment, disposing of the inner layers within the outer layers comprises providing the plurality of compressed cores of the high molecular weight water-soluble polymer within the centers of the plurality of rings.

In accordance with another embodiment of the method of the present invention, preparation of the inner layer comprises compressing at least one core of the high molecular weight water-soluble polymer and including disposing the at least one core of the high molecular weight water-soluble polymer in at least one of the plurality of rings, and including providing at least one core of the low molecular weight water-soluble compound and disposing the at least one core of the low molecular weight water-soluble compound in another of the plurality of rings.

### DESCRIPTION OF THE DRAWINGS

FIG. 1 is a graph showing long-term activity of a composition containing the carrageenans of the present invention. Mice were challenged with a 95-100% infectious dose of HSV-2 at various time intervals after application of the composition. The composition retains some level of activity against HSV-2 even after 24 hours. This suggests that a woman could be protected even if considerable time elapsed between use of the composition and coitus.

FIG. 2 is a graph of Southern Blot hybridization of RT PCR products from RNA extracted from the spleens. Lane 2 and 3 are positive controls. Lanes 4 to 8 are from mice that were pretreated with a composition containing the carrageenans of the present invention, 5 minutes before viral challenge. Lanes 9 to 14 are from mice inoculated vaginally with HIV.

FIG. 3 is a bar graph showing p24 (HIV) concentration versus concentration of a composition containing the carrageenans of the present invention, another composition of the present invention that contains a complex of the carrageenans and a water-soluble zinc salt (“zinc-carrageenan”), and liposulfonic acid (LSA).

FIG. 4 is a graph showing comparison between a composition of the present invention containing the carrageenans and LSA, and a composition of the present invention containing the carrageenans, in the HSV-2/Mouse system. The results show that the composition containing LSA and the carrageenans is more efficacious than a composition containing the carrageenans alone.

FIG. 5 is a plot of the percent inhibition by LSA of viral replication as measured by p24 ELISA.

FIG. 6 is a graph of the efficacy of a composition containing the carrageenans of the present invention, and another composition of the present invention that contains zinc-carrageenan, in preventing plaque formation of HSV-2 in Vero cells as a function of dose.

FIG. 7 is a graph showing the efficacy of a composition containing the carrageenans of the present invention, and another composition of the present invention that contains zinc-carrageenan, in protecting mice from infection from HSV-2, following vaginal Challenge.

FIG. 8 is a graph showing the comparison of long-term activity of a composition of the present invention containing zinc-carrageenan compared to two blown products, Conceptrol and Advantage S, at a viral challenge dose of 10⁴ or 100% infection dose of HSV-2.

FIG. 9 is a graph showing protection against viral challenge by a composition containing the carrageenans of the present invention another composition of the present invention that contains zinc-carrageenan.

FIG. 10 is a graph of the amount of Nestorone released from a composition containing the carrageenans of the present invention.

FIG. 11 is a bar graph comparing the effectiveness of various dilutions of carrageenan compositions of the present invention in protecting mice from infection by HSV-2. Results show that even when the carrageenans are diluted 1:200, they still were able to provide 40% protection from infection.

FIG. 12 is a top elevational view of a portion of the vaginal ring in accordance with the present invention.

FIG. 13 is a side elevational view of the vaginal ring shown in FIG. 12.

FIG. 14 is a top elevational view of the portion of the vaginal ring shown in FIG. 12 including a wire for creating a groove therein.

FIG. 15 is top elevational view of the portion of the vaginal ring shown in FIG. 12 including the groove produced therein.

FIG. 16 is a side elevational view of the completed vaginal ring of the present invention including the inner groove containing an inner core of compressed material.

FIG. 17 is a top elevational view of a completed vaginal ring in accordance with the present invention including a plurality of apertures through the surface thereof for connection with the groove therein.

FIG. 18 is a side perspective view of another embodiment of the vaginal ring of the present invention.

FIG. 19 is a top devotional view of another embodiment of the vaginal ring of the present invention.

FIG. 20 is a top, elevational view of another embodiment of the vaginal ring of the present invention.

FIG. 21 is a top, elevational view of a mold for producing one embodiment of the vaginal ring of the present invention.

### DETAILED DESCRIPTION OF THE INVENTION

The polyanionic microbicides used in the compositions and in the intravaginal rings (IVRs) of the present invention are microbicides which interfere with viral attachment so as to reduce HIV transmission across mucosal surfaces. These polyanionic microbicides include compounds such as PRO2000, Buffergel, dextrin sulfate, cellulose sulfate, and most preferably the carrageenans.

The carrageenans present in compositions of the present invention include a lambda carrageenan. To the extent that non-lambda carrageenans are present (in which the case the carrageenan component of the compositions may be referred to as “the carrageenans” or the “carrageenans mixture”), the carrageenans mixture contains at least about 50% (and preferably at least 50%) of lambda carrageenan, based on total dry weight of the carrageenans in the composition. In more preferred embodiments, the amount of lambda carrageenan is at least about 75, 76, 77, 78, 79, 80, 81, 82, 83, 84, 85, 86, 87, 88, 89, 90, 91, 92, 93, 94, 95, 96, 97, 98, or 99% of the total dry weight of the carrageenans (i.e., lambda and non-lambda carrageenans). Other preferred amounts are at least 75%, at least about 85%, at least about 95%, about 85 to about 99%, and from about 94 to about 97% lambda carrageenan.

Lambda carrageenan is commercially available (FMC Corp., Philadelphia). Alternatively, lambda carrageenan can be produced from diploid (sporophyte) seaweed plants e.g., Gigartina radula, Gigartina skottsbergii, Gigartina chamissoi, Gigartina stellata, Iridaea cordata, Chondrus chrispus and Sarcothalia crispata. Isolation of the carrageenan from the seaweed is conducted in accordance with standard techniques. For example, the seaweed is separated, cleaned and then dried. Lambda carrageenan is extracted in hot dilute sodium hydroxide, yielding a paste that contains as much as 4% concentration of lambda carrageenan. The resulting paste is clarified by centrifugation and filtration to yield a clear, lambda carrageenan solution. Water is removed by any combination of evaporation, alcohol precipitation or washing, and drying.

The remainder of the carrageenans in compositions of the present invention may include at least one non-lambda carrageenan. By “non-lambda carrageenan”, it is meant any carrageenan other than lambda carrageenan, such as kappa-carrageenan, iota carrageenan kappa-II carrageenan (which contains kappa and iota carrageenans), mu carrageenan, and nu carrageenan. Non-lambda carrageenans are also available commercially (e.g., FMC Corp.) or may be extracted from seaweed in accordance with standard techniques. For example, kappa-II carrageenan is also naturally present in the species of seaweed described above. In preferred embodiments, the non-lambda carrageenans include kappa carrageenan, iota carrageenan, and kappa-H carrageenans, and mixtures of any two or more thereof. In more preferred embodiments, the non-lambda carrageenan includes kappa-TI carrageenan. In preferred embodiments, the non-lambda component of the carrageenans constitutes less than about 1, 2, 3, 4, 5, 6, 7, 8, 9, 10, 11, 12, 13, 14, 15, 16, 17, 18, 19, 20, 21, 22, 23, 24 or about 25% of the total dry weight of the carrageenans. In more preferred embodiments, the non-lambda component is about less than about 25%, less than about 15%, less than about 5%, about 1 to about 15%, or about 3 to about 6% of the total dry weight of the carrageenans. In other preferred embodiments, the carrageenan mixture is substantially or entirely free of dextrose, an ingredient commonly found in carrageenans used in the food industry.

In order to provide an antimicrobial effect, the lambda carrageenan or the carrageenans are generally present in amounts of about 1 to about 5%, based on total weight of the composition. In preferred embodiments, the carrageenans are present in amount of about 3% by total weight of the composition. By “antimicrobial” or “antimicrobial effect”, it is meant that the composition inhibits or reduces the likelihood of transmission of a sexually transmitted infection caused by a bacterium, another microbe or a virus. The compositions of the present invention useful in protection against sexually transmitted infections e.g., by inhibiting infection by HTV, HPV, HSV-2 and Neisseria gonorrhoeae. On the other hand, the terms “antimicrobial” and “antimicrobial effect” are not meant to convey, imply or be limited to any particular means by which the inhibition of transmission of the infection is accomplished. Without intending to be bound by any particular theory of operation, it is believed that the carrageenans non-specifically bind to virus, bacteria and other microbes that are etiological agents of STIs, thereby blocking receptor sites. Compositions containing the lambda carrageenan or the carrageenans in amounts less than 1% or greater than 5% may be used so long as that they provide an antimicrobial effect and retain vaginal acceptability. By “vaginal acceptability”, it is meant that the rheological properties such as viscosity of composition allow it to be used for its intended propose (e.g., the composition maintains a viscosity so that it can be applied by the user and be retained in the vaginal vault, as well as providing aesthetic properties such as being substantially odorless, smoothness, clarity, colorlessness and tastelessness). The viscosity is selected so as to enable the composition to evenly coat the epithelial lining of the vaginal vault. In general, the viscosity of the compositions is about 10,000 to about 50,000 cP, preferably about 20,000 to about 50,000 cP, and more preferably about 30,000 to about 50,000 cP. Carrageenan has a continuum of molecular weights. In general, the carrageenan mixtures of the present invention may have a molecular weight of up to about 2×10⁶ daltons with less than about 1% of carrageenan molecules having an average molecular weight of 1×10⁵ daltons (as determined by gas permeation chromatography and light scattering). More particularly, a lambda carrageenan in the invention has a weight average molecular weight of about 600,000 to about 1,200,000 daltons. This physical property imparts non-absorbability to the final formulation that in turn provides prolonged anti-microbial activity.

Among the other polyanionic microbicides, other than the carrageenans. Which can also be used in the compositions of the present invention, is PRO2000. This microbicide is a vaginal microbicide for my prevention. In addition, other such polyanionic microbicides include Buffergel, a microbicidal spermicide which provides buffering activity to maintain the mild, protective acidity of the vagina in the presence of semen. In addition, dextrin sulfate, a polyanion which blocks the entry of HIV at the surface of the cell, and cellulose sulfate can also be utilized therefor.

The composition further contains a physiologically acceptable pH controlling agent such as phosphate buffered saline (PBS). In addition to stabilizing the pH of the composition (e.g., at a level of about 3.5 to about 8.5, and preferably about 5.8 to about 7.2, such as from about 6.8 to 7.2), the pH controlling agent prevents or reduces any change of the change in the composition once it is introduced into the body where the pH can vary significantly. Vaginal pH can range between 3.5 to 5.5. Thus, the presence of the pH controlling agent extends the antimicrobial effect of the carrageenans. The compositions include from bout 0.001% to about 1.0% of the pH-controlling agents. The compositions formulation may further contain other active agents and/or inert ingredients, depending upon the intended use (as described below).

The carrageenans of the present invention provide several other benefits. They remain stable if exposed to freezing, ambient, or boiling temperatures. The mixture is compatible with the human vaginal environment. Without intending to be bound by any particular theory of operation, it is believed that the carrageenans are compatible with the human vaginal environment and do not act as a substrate or otherwise cause or stimulate growth of natural vaginal flora, nor are they toxic so as to disrupt the natural floral balance in the vagina. Aside from the properties attributable to the carrageenans of the present invention, their antimicrobial activity extends over a period of time because they are not systemically absorbed or degraded to any absorbable by-products detrimental to humans.

Another aspect of the present invention is directed to a complex between a water-soluble metal salt and the carrageenans. In preferred embodiments, the metal salt is a zinc salt (and the antimicrobial composition is referred to as “zinc carrageenate”). Zinc is an inhibitor of such sexually transmitted pathogens as HIV and HSV-2. Zinc acetate and zinc sulfate have been shown to inhibit HIV infection in cell culture, and HSV-2 in both cell culture and laboratory animals. Zinc salts have been shown to be effective in blocking infection by HIV in vitro³⁹, foot-and-mouth virus, human rhinovirus, influenza A and B, semliki forest virus and sindbis virus⁴⁰. Haraguchi, et al.³⁹ found that zinc chloride, cadmium acetate and mercury chloride inhibited HIV-1 production as assayed by p24 ELISA and RT. Zinc chloride did not exhibit significant cytotoxicity when present in concentrations of up to 550 μg/mL.

Water-soluble zinc salts useful in the present invention include both inorganic salts and organic salts that exhibit anti-microbial properties without causing unacceptable irritation when used in accordance with the present invention. Preferred water-soluble zinc salts include zinc acetate, zinc propionate, zinc butyrate, zinc formate, zinc gluconate zinc glycerate, zinc glycolate zinc lactate, zinc sulfate, zinc chloride, and zinc bromide. ZirSO₄, ZnCl₂, ZnBr₂, Zn(Ac)₂, etc. Copper and silver counterpart salts are also useful in the present invention provided that they are non-irritating in vivo and do not cause degradation to any absorbable by-products detrimental to humans. The compositions of this invention will thus include between about 0.03% and 1.5% of the water-soluble metal salts, preferably from about 0.3% to 1.0%. The anti-microbial activity of the composition is greater than a formulation containing the carrageenans as the only anti-microbial agent. In embodiments of the present invention with specific zinc salts, there is a significant increase in anti-microbial activity. Without intended to be bound by any particular theory of operation, it is believed that the anti-microbial activity of the formulation is enhanced because the rate at which the metal salt is absorbed by the body is relatively controlled and at the same time, the irritation of the metal salt is reduced.

The complexes of the present invention may be prepared by standard processes whereby the metal ions replace cations that are naturally present on the backbone of the polysaccharide. For example, zinc carrageenan (which refers to a complex between zinc cations and the carrageenans of the present invention) is a compound synthesized by a procedure whereby zinc (II) is non-covalently attached to the sulfate groups of the carrageenans. Carrageenan is a polysaccharide consisting of repeating D-galactose and 36-anhydro D-galactose units arranged in a linear fashion. The polymer is highly sulfated having. 3 S0₃ groups per each disaccharide unit. The binding of zinc to the carrageenans is accomplished by a chemical process developed to replace sodium bound to native carrageenan with zinc. An aqueous solution of a highly soluble zinc salt (such as zinc acetate) is used in this process as a source of zinc cations. The carrageenans are dialyzed against a concentrated solution of zinc acetate allowing positively charged zinc ions to diffuse and complex with the negative sulfate groups of the carrageenans. Excess of zinc is then removed by dialysis against water.

The inclusion of a complex of zinc II metal cations with the carrageenans in the present invention can be achieved by the use of zinc II carrageenate. Zinc carrageenate is synthesized by substitution of the natural carrageenan cations (sodium, potassium, calcium) by zinc cations. Zinc carrageenate is traditionally prepared by dialysis of a solution of carrageenan against a concentrated solution of zinc II acetate. Excess zinc cations are then removed by dialysis against water, before concentrating, and for example, freeze drying. The use of zinc II carrageenate can avoid the use of anions such as lactate or acetate in the present invention.

Another process entails (a) soaking the carrageenans in about a 2.5% zinc lactate (or other suitable soluble zinc salt) in 50:50 alcohol:water liquor for two hours, (b) separated, and (c) washed with alcohol before drying. Steps (a) through (c) may need to be repeated several times to achieve the desired metal content in the carrageenans. Two cycles are normally required to achieve over 50% zinc carrageenan on an equivalent basis.

The above procedures generate a compound, which is water soluble and active against enveloped viruses such as HIV and HSV-2. Unlike inorganic or simple organic zinc salts, zinc carrageenan maintains the preferred rheological properties and possesses a high molecular weight (up to 2,000,000 Da) making it amenable to be formulated into a vaginal product, which is non-irritating and not absorbed. The composition is referred to as a “complex” due to the presence of molecular interactions between the metal and the carrageenans that disfavor or discourage its dissociation to free metal cations. The present complexes of a metal salt and a negatively charged sulfated-polysaccharide complex are distinct from mixtures of water-soluble metal salts and carrageenans in terms of their physical, chemical and/or anti-microbial properties

In another aspect of the present invention, applicants have discovered that a combination of the complex between a water soluble metal salt and carrageenans along with specific antiretroviral agents provides unexpected advantages and synergistic properties. Most particularly, this particular combination of ingredients has been found to provide unexpected results in terms of inhibition of sexually transmitted infections and particularly in blocking vaginal SHIV-RT infections (simian/human immunodeficiency virus-reverse transcriptase). Antiretroviral agents are drugs used for the treatment of infection by retroviruses, primarily HIV. There are a number of different classes of antiretroviral drugs which act at different stages of the HIV life cycle, and these include, for example, non-nucleoside reverse transcriptase inhibitors (NNRTIs), nucleoside reverse transcriptase inhibitors (NRTIs), integrase inhibitors, fusion inhibitors, and CCR5 antagonists.

NNRTIs are compounds which attach themselves to reverse transcriptase and prevent the enzyme from converting RNA to DNA so that HIV's genetic material cannot be incorporated into the healthy genetic material of cells and the cells can be prevented flour producing new viruses. These include drugs such as nevirapine, delavirdine, efavirenz, etravirine, MIV-150, MIV-160. MIV-170, dapivirine (TMC-120), and UC-781. Most preferably the NNRTI would be MIV-150, an NNRTI developed by Medivir for use as an antiviral therapeutic. MIV-150 is a tight-bonding HIV-RT enzyme inhibitor characterized by a rapid formation and slow dissociation rate that is effective at inactivating Chemical isolates of HIV at very low concentrations. In a preferred embodiment of the present invention, applicants have thus discovered that the specific combination of the carrageenans of this invention with water-soluble metal salts, preferably such as zinc, as well as the NNRTIs such as MIV-150, are effective in totally blocking vaginal SHIV-RT infection. Furthermore, this specific combination has been found to be significantly more effective than the individual combination of the carrageenans with water-soluble metal salts such as zinc; or the carrageenans with NNRTIs, or the carrageenans themselves. In a preferred embodiment, the combination of the carrageenans of the present invention, the water-soluble metal salts, and the NNRTI preferably include the carrageenans discussed above, including lambda carrageenan in amounts of at least about 50% by dry weight of the carrageenans with the remainder of the carrageenans being at least one non-lambda carrageenan, and most preferably a combination of 95% lambda carrageenan and 5% kappa carrageenan, with the overall composition in the form of a gel including between 1% and 5% carrageenan, preferably about 3% carrageenan: the compositions include the water-soluble metal salts preferably comprising zinc salts, most preferably in the form of zinc acetate or zinc lactate, including from about 0.1 wt. % and 1.5 wt. % of the metal, such as zinc, in the overall composition, most preferably about 0.3 wt. % thereof, and include the NNRTIs, most preferably MIV-150, in amounts of from between 5 μM to 5,000 μM (or 0.000185% to 0.185%), and preferably from between 10 μM and 250 μM (or 0.00074% to 0.00925%) of the NNRTI, such as MIV-150, most preferably about 50 μM (or 0.00185 of the MIV-150.

NRTIs are compounds which are incorporated into the DNA of the virus to stop the building process. They thus result in incomplete DNA that cannot create a new virus. These include drugs such as abacavir, tenofovir and zidovudine.

It has been found by applicants that the NNRTIs and the NR Ifs exhibit specific unexpected properties when used in the compositions of the present invention.

In another aspect of the present invention, lignosulfonic acid (“LSA”) is combined with a lambda carrageenan or the carrageenans (referred to herein as LSA-carrageenan), to achieve an enhanced anti-microbial effect. LSA is commercially used as an industrial stabilizer, dispersing agent, and strengthener. It is also used as a source of bulk-fiber in cattle feed, and as an emulsifying and dispersing agent in processing certain foods for human consumption. It exists in the cell walls of higher plants. The cell wall fibers are generally made of the polysaccharide, cellulose, the most abundant polysaccharide on earth. In addition to cellulose, the secondary cell wall contains another very abundant material called lignin which is the polysaccharide that makes plants stiffer. By cooking wood chips in a solution of calcium bisulphate under heat and pressure, lignin is converted to a water soluble lignosulfonic acid (LSA) solution known as spent sulfite liquor^{31,32}. It is a low molar mass compound with an average molecular weight of approximately 5000 Daltons. Because lignins are very complex natural polymers with many random couplings, the exact chemical structure is not known, but it is considered to be that of a sulphonated polymer in which the basic unit is a propylbenzene structure similar to that of coniferyl alcohol. The usefulness of commercial lignosulfonate comes from its dispersing, binding, complexing and emulsifying properties. The aromatic ring structure of hgnosulfonic acid confers on plants the ability to resist attacks from microbes. LSA has been shown to have in vitro anti-HIV activity.

Formulations comprising the carrageenans and LSA can be prepared by adding LSA to the carrageenans, generally in an LSA-total carrageenan weight ratio of from about 20:1 to about 1:20. As in the case of compositions containing a metal salt, a solid buffer salt can be mixed with the carrageenans, usually in a weight ratio of from about 1:1 to about 10:1. The resultant mixture is then solubilized in an aqueous solution. The pH of the carrageenan-LSA formulation may then be adjusted to be from about 6.0 to about 8.0 by adding an acid such as HCl, or a base such as NaOH. LSA in aqueous solutions yields a tan to brown coloration. The intensity of which increases proportionally with the concentration used. Thus, a whitening agent such as titanium dioxide may be included in the composition. In general, the whitening agent is present in an amount of about 0.1 to about 3.0% based on total weight of the composition. The whitening agent may also contribute to the antimicrobial effect.

Without intending to be bound by any particular theory of operation, it is believed that aside whatever anti-viral activity LSA exerts on its own, LSA also functions as a dispersing agent for the carrageenans, and disentangles and elongates them, thus creating greater density of this material and greater anti-microbial potency. On the other hand, the carrageenans provide the preferred rheological properties necessary for acceptable and effective vaginal (and even rectal) administration, which cannot be achieved by LSA in and of itself because it is rather watery in nature. In some embodiments, the combination of the carrageenans and LSA acts synergistically in preventing or inhibiting sexually transmitted infections.

Compositions of the present invention may also contain a vaginally administrable drug in the aqueous formulation along with the pH controlling agent and the lambda carrageenan or the carrageenans. Preferred drugs are contraceptive agents, such as steroid hormones, disclosed in Saleh, et al., U.S. Pat. No. 5,972,372 (“Saleh”), the disclosure of which is hereby incorporated by reference. Examples of contraceptive agents useful in the present invention include progestins. ACTH, androgens, estrogens, gonadotropin, human growth hormone, menotropins, progesterone, progestins (e.g., levonorgestrel, norethindrone, 3-keto-desogestrel and gestodene), progestogen, urofollitropin, vasopressin and combination thereof. Preferred agents include progestational compounds (e.g., norethindrone acetate and NESTORONE™ (“NES”), (i.e., 16-methylene-17.alpha.-acetoxy-19-norpregnene-3,20-dione)), and progestins (e.g., levonorgestrel (LNG)).

A preferred contraceptive agent is Nestorone 16-methylene-17α-acetoxy-19-norpregn-4-ene-3,20-dione (hereinafter “NES”), which has been identified in the literature as ‘ST-1435 ’. In comparative studies using the classic bioassay of measuring progestational potency. NES was found to have progestational activity 100 times higher than that of progesterone and 10 times higher than that of levonorgestrel⁵³. Therefore, smaller amounts of NES are required to achieve ovulation inhibition. This potency combined with a lack of androgenic, estrogenic and glucocorticoid-like (hepatic glycogen deposition) activity and the lack of effects on lipid or clinical chemistry parameters, confer special advantages for the use of NES in contraceptives⁵³⁻⁵⁵. However, NES has been shown to undergo rapid metabolism and inactivation upon oral administration making it suitable for use in nursing women when given via implants or vaginal rings^{56,57}. A preferred delivery dose of NES when combined with the K/λ carrageenan mixture in gel form is between about 75 and about 100 μg per day, which will reach plasma levels of NES around 200 pmol/L and achieve good bleeding patterns during menses. Other preferred vaginally administrable drugs include agents for hormone replacement therapy such as estrogenic substances (e.g., ethynylestradiol) and other steroidal compounds.

Without intending to be bound by any particular theory of operation, it is believed that the carrageenans possess a dual function of imparting microbicidal properties while providing a prolonged release delivery system for a contraceptive agent or agent for hormone replacement therapy, thus enhancing the activity of the agent.

Any of the compositions described herein may further contain at least one physiologically inert ingredient, such as a physiologically acceptable preservative. Preservatives include alkyl esters of para-hydroxybenzoic acid, such as methyl paraoxybenzoate, propyl paraoxybenzoate, hydantoin derivatives, parabens, such as methyl when, propioniate salts, triclosan tricarbanilide, tea tree oil, alcohols, farnesol, farnesol acetate, hexachlorophene and quaternary ammonium salts, such as benzolconjure, zinc and aluminum salts, sodium benzoate, benzyl alcohol, benzalkonium chloride and chlorobutanol. In general, the preservative is present in an amount up to about 0.3% based on the total weight of the composition. In addition to inhibiting the growth of microorganisms that may be introduced inadvertently during manufacturing, the preservative prevents any deleterious effects that might occur to the active agents in the composition due to the presence of normal body flora once the composition is introduced into the body. This will prolong the length of time that the active agents in the composition remain active.

In preferred embodiments, the compositions of the present invention containing the carrageenans as the sole antimicrobial agent, with or without a vaginally administrable drug, and the rings, foams, films, suppositories and gels that contain an additional antimicrobial agent such as the cationic metal salt or LSA, are administered vaginally. The present invention also includes rectal administration. The compositions may be suitably formulated e.g., into gels, creams, foams, films and suppositories, in accordance with standard techniques in the pharmaceutical industry. The gel formulations can be administered prior to sexual activity such as intercourse, usually within about one hour before such time. The application of the carrageenan-based formulation in human prevents or inhibits transmission of a sexually transmitted infection (STI), such as Neisseria gonorrhoeae, human papillomavirus, HSV-2 and HIV.

In another aspect of the present invention, a particularly preferred method of administering these and many other such compounds has been discovered. Thus, a novel intravaginal ring (IVR) has been discovered, particularly for the administration of water-soluble polymers, and preferably high molecular weight water-soluble polymers, including but not limited to carrageenan, and preferably the particular carrageenans and carrageenan combinations with metal salts, such as zinc salts, as discussed above. While this new IVR can thus be used with carrageenan as well as many other high molecular weight water-soluble polymers, which will be discussed below, the specific compounds such as carrageenan, and the other high molecular weight water-soluble compounds discussed above can be used, but do not need to be used in the form of a gel or the like. Thus, a portion of the disclosure of the compounds of the present invention will now be unnecessary when they are used in these novel IVRs. In particular, and discussing carrageenan itself, the carrageenan compositions hereof can be used in a dried form, and not in the form of the gels described in this application. While most of the disclosure above with respect to the specific compounds useful in the present invention will apply to the disclosure of these IVRs, the specific details of compounding these molecules, such as into the form of gels and the like, is not necessarily required for use therein. On the other hand, administration of the water-soluble polymers of the present invention can also include low molecular weight water-soluble compounds. In this case, however, it is necessary to combine these low molecular weight water-soluble compounds with an elastomeric polymer which enables the sustained release of the low molecular weight water-soluble compound therefrom. These elastomers include, for example, polyurethane elastomers such as the thermoplastic silicone polyether urethane known as PURSIL.

In the discussion of this aspect of the present invention, and in particular the novel IVR's discussed herein, the term “active agent” has been utilized. This is intended to broadly refer to each of the water soluble and water insoluble compounds discussed herein, including the high molecular weight water soluble polymers, and the low molecular weight water insoluble and water soluble compounds. Each of the specific such compounds discussed herein thus have pharmaceutical utilities, as drug compounds., antimicrobials, antiretrovirals, antibiotics, etc.

The novel IVRs which have now been discovered can thus directly employ the preferred high molecular weight water-soluble polymers in a compressed or compacted form, directly in or associated with the IVR itself. It is now possible, using these devices, to deliver these high molecular weight water-soluble compounds at essentially zero order release rates. Furthermore, it is now possible to do so without utilizing a separate plastic pod or container for these high molecular weight compounds, or to encapsulate or otherwise cover these high molecular weight water-soluble compounds with polymeric layers or coatings which would prevent or interfere with the release of these high molecular weight compounds therefrom.

In accordance with the present invention, these water-soluble compounds, and most particularly the large, high molecular weight water-soluble polymers, are incorporated directly into the IVRs themselves. A number of methods of doing so are disclosed in this application. In general, however, these high molecular weight compounds are incorporated directly into the IVR ring structure and/or are pelletized, with the pellets being incorporated into the ring structure and/or into a complementary film or sheath of non-water-swellable elastomer, either the same as that of the ring structure itself, or another such non-water-swellable elastomer sheet or film which is compatible with that of the ring structure. In effect, this becomes a mere extension of the ring structure itself, and in total provides an outer layer of non-water-swellable elastomer which encompasses the inner layer of high molecular weight water-soluble polymer. These high molecular weight water-soluble polymers are thus fully encapsulated, either by the ring structure itself or such a structure associated with the ring structure, and since they are not able to readily diffuse through the non-water-swellable elastomer layer itself, they can only be delivered through an aperture or apertures which are formed through that ring structure (the outer layer), whose size and/or numbers can be selected in order to obtain optimal release results.

As for these high molecular weight water soluble polymers themselves, these can be considered to be macromolecules including sulfated polysaccharides such as carrageenan, particularly lambda carrageenan, and particularly the carrageenans discussed above, which in a preferred embodiment includes, at least about 50% lambda carrageenan on a dry weight basis, with the remainder of the carrageenans being at least one non-lambda carrageenan. These macromolecules can also include proteins, including but not limited to lectins, such as griffithsin, glycoproteins, peptides/polypeptides, peptide hormones, such as insulin, nucleic acid derivatives, including oligonucleotides and aptamers, glucoaminoglycans (GAGs), HTV-1 envelope proteins, HSV envelope proteins, therapeutic antibodies and the line, polyacrylic acids, like carbopol, as well as polypyrroles. In general, by high molecular weight these macromolecules are meant to have molecular weights between about 1,000 and 10,000,000 Da, preferably between about 2,000 and 5,000.000 Da, and most preferably between about 5,000 and 3,000,000 Da.

As is also discussed above, the inner layer of high molecular weight water-soluble polymer is preferably prepared by simply compressing these compounds under significant pressure. The degree of compression itself can effect the release rates obtained, but as noted above, the present invention can lead to substantially zero order release rates. In general, such pellets of these high molecular weight water-soluble polymers, can either be utilized alone or in combination with other ingredients, such as the low molecular weight water-soluble compounds discussed herein.

These low molecular weight water-soluble compounds include the water-soluble metal salts discussed above, including metal salts such as zinc salts. These zinc salts include both inorganic and organic salts which exhibit antimicrobial properties, preferably including zinc acetate, zinc sulfate, zinc lactate, and the like. Other such water-soluble compounds, include compounds such as probiotics, including lactobacilli. HIV-1 envelope proteins, HSV envelope proteins, therapeutic antibiotics and the like, pseudovirions such as HVP, HIV, HSV and HSV pseudovireones and antigens and/or other immunogens and combinations of any two or more thereof.

The low molecular weight water-insoluble molecules of the present invention can include the NNRTIs discussed in this application, including drugs such as navirapine, delavirdine, efavirenz, estrairine, MIV-150, MIV-160, MIV-170, dapivirine (TMC-120), and UC-781, and most preferably the MIV-150 which is highly preferred in combination with the complex between a water-soluble metal salt and the water-soluble carrageenans discussed above. In any case, the inner layer of water-soluble compounds hereof are preferably free to diffuse out of these IVRs upon contact with water. In a preferred embodiment, they are neither contained within a separate protective container or pod nor covered by a separate polymeric coating, cover or sheath. However, in one embodiment they are contained within a sheath, which is preferably identical to or comparable to the non-water-swellable elastomer of the IVR itself, primarily for the purpose of physically separating the inner layer from the active agents in the IVR. They are thus simply prevented from diffusing out of the IVR by means of the non-water-swellable elastomer of the IVR itself, or with the non-water-swellable elastomer and a second elastomer sheath, either water-swellable or non-water-swellable, and thus can only be released therefrom through the holes or apertures in the IVR or the IVR and the elastomeric sheath utilized in accordance with this invention.

Those high molecular weight water-soluble compounds, such as the carrageenans discussed above, can preferably be used in a dry form without the need for admixing additional components, since they are compressible in that form. This is also true for the zinc-carrageenan mixtures discussed herein. However, even with certain of the zinc-carrageenan mixtures with high zinc contents, this may begin to negatively impact the compressibility of these compounds. Furthermore, there are others of the high molecular weight water-soluble compounds discussed above which will not be amenable to simple compression and under compression will not compact sufficiently to maintain their integrity in the manner required by this invention. In those cases, it is therefore possible to add an additional component acting as a binder to provide these high molecular weight water-soluble compounds with sufficient compressibility to be compacted for use in the present invention. These compounds are the same compounds discussed above in connection with blending the low molecular weight water-soluble polymers within the inner layer of the present invention, such as the hydrophilic polyurethane binders discussed above. These additives or binders can thus both be used to create a necessary compressibility when used in connection with the high molecular weight water-soluble polymers, but also to be used to enable sustained release of the low molecular weight water-soluble compounds when used in combination therewith.

In general, when the water-soluble compounds of the present invention comprise low molecular weight water-soluble compounds, it is necessary to combine these compounds with an elastomeric polymer. In one embodiment, the elastomeric polymers comprise hydrophilic or water-swellable elastomers. These include aliphatic thermoplastic urethanes, such as the poly(ether urethane)s or silicone poly(ether urethane)s disclosed in International Application No. WO 2013/013172, in Paragraphs [00021] through [00023], which are incorporated herein by reference thereto. These include hydrophilic poly(ether urethane)s such as the TECOPHILICS thermoplastic silicone-polyether urethanes such as the PURSIL ALs, thermoplastic polyether urethanes such as the ELASTHANEs and PELLETHANEs, and the like. Also, as disclosed in WO 2013/013172, these can include non-swellable or hydrophobic elastomers, such as poly (ether urethane)s and the TECOFLEXs, all of which are incorporated herein by reference thereto.

In addition, the inner layer of high molecular weight water-soluble polymer can also include an amount of low molecular weight water insoluble molecules, which can also be included in the outer layer of water-impermeable polymeric compound. These low molecular weight and primarily water-insoluble compounds include intravaginally administrable drugs such as cervical anesthetics, contraceptives, anti-endometriosis drugs, estrogen receptor modulators, preterm labor drugs, overactive bladder drugs, morning sickness drugs, osteoporosis drugs, antimicrobials, vaccines, and the like. Thus, useful intravaginally administrable substances include, but are not limited to, cervical anesthetics such as lidocaine, contraceptives such as 17α-ethinyl-levonogestrel-17b-hydroxy-estra-4,9,11-trien-3-one, estradiol, etonogestrel, levonogestrel, medroxyprogesterone acetate, NESTORONE, norethindrone, progesterone, estrogen receptor modulators such as RU-486, anti-endometriosis drugs such as Terbutaline, antivirals such as acyclovir and gancyclovir; blood flow increasing drugs like Sildenafil: labor-inducing drugs like misoprostol; preterm labor drugs like indomethacin; overactive bladder drugs like oxybutynin; morning sickness drugs such as Bromocriptine; osteoporosis drugs like human parathyroid hormone; drugs and/or substances for vaginal dryness such as glycerol and/or other lubricating or hydrating substances.

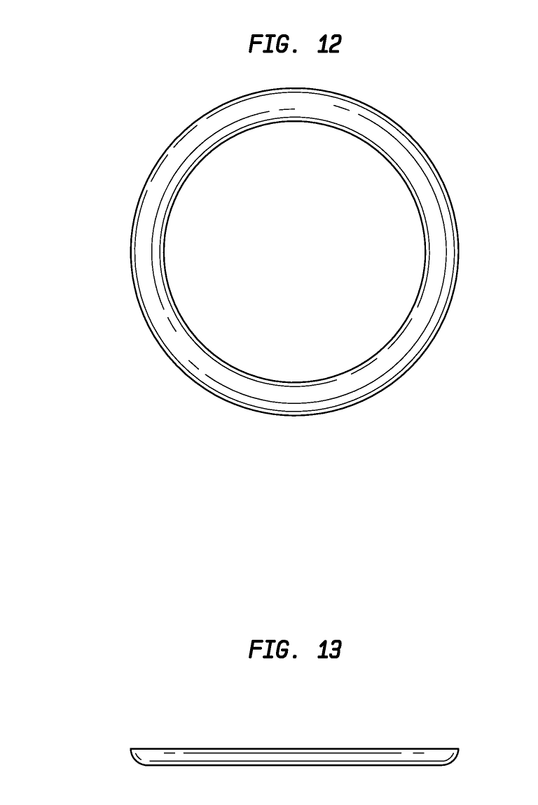

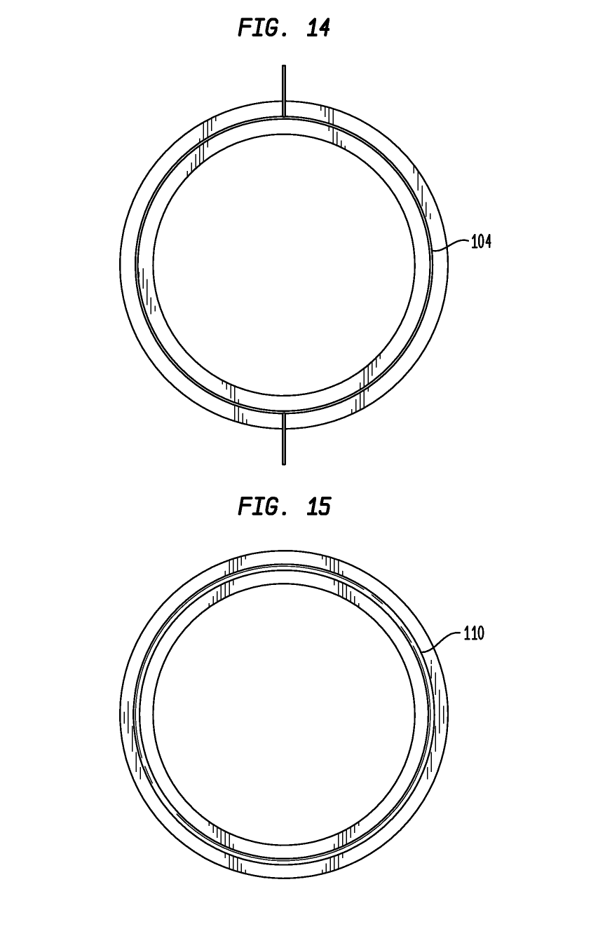

The vaginal rings of the present invention can be more fully appreciated with reference to the drawings. The IVRs themselves are well brown and are generally made from non-water-swellable elastomers, which can be either thermosetting or thermoplastic elastomers. Thermoplastics include, but are not limited to, polyurethanes and ethylene vinyl acetate (EVA). Thermosetting resins include, but are not limited to, the class of silicone polymers. IVRs can also be made from a mixture of any two or more polymers and generally have a toroidal shape. In one embodiment of the present invention, the ring includes an inner layer of low and/or high molecular weight water-soluble polymer embedded within the ring itself. One method of producing this product is as follows: One half of such a ring 100 is shown in FIGS. 12 and 13. It is generally injection molded, and has a bottom surface 101 having the shape of the ring and an upper planar surface 102. In order to embed the high molecular weight water-soluble polymer within the ring, a groove is produced in the upper surface 102. One method of doing this is to embed a heated wire 104, such as a brass wire, onto the upper surface 102, and applying pressure thereto in order to create a groove 110 in the upper surface 102 of the ring 100. This is shown in FIG. 14, and the groove 110 is then created in the upper surface 102 of the ring 100. It is then possible to fill the groove 110 with compacted high molecular weight water-soluble polymer, preferably in the form of a pellet or pellets compressed into the groove itself, or with a low molecular weight water-soluble compound, which is preferably compounded with a hydrophilic or hydrophobic elastomeric polymer which enables the sustained release of the low molecular weight water-soluble compound therefrom.

An alternate method of preparing the groove 110 can be seen by using the mold shown in FIG. 21. The mold half on the left side of FIG. 21 shows mold half 116 which includes a number (six in this case) of ring molds 118, each connected to a central source of polymer for filling the mold. The right hand mold 120 shown in FIG. 21 includes a circular projection 122 corresponding to each of the ring molds 118. Thus, by injecting the polymer into ring molds 118 and closing the mold halves (116, 120), the projections will project into the molten polymer and thus create a groove in each of the ring molds in this manner.

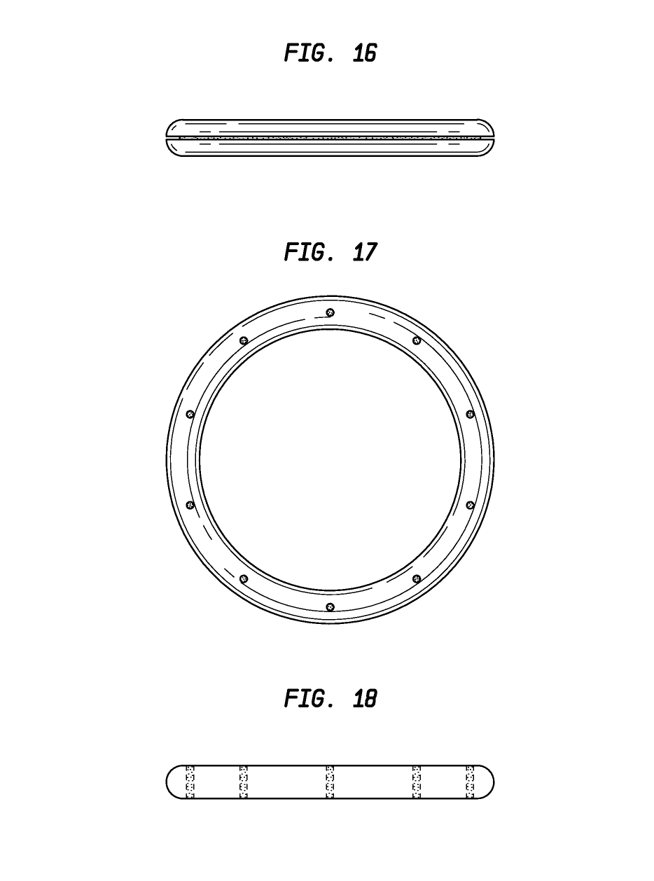

In either case, the result is half of a ring including a groove 110 in the upper surface 102 thereof. The ring itself is then completed by producing a second half of the ring identical to that Shown in FIGS. 12 and 13 by injection molding in the normal manner where the second half of the ring is injection molded over the first half Molding the entire ring thus produces an IVR which contains an inner layer of either the compressed high molecular weight water-soluble polymer or the low molecular weight water-soluble compound admixed with an elastomeric polymer, imbedded within the ring itself, as shown in FIG. 16.

In order to produce the final product, it is then only necessary to create at least one aperture 124 in the IVR 123 projecting through one of the surfaces of the IVR and extending into contact with either the compressed high molecular weight water-soluble polymer or the low molecular weight water-soluble compound 121 therein, thus providing a path for release of the water-soluble polymer from the IVR. In this embodiment, and as is shown in FIG. 17, a plurality of apertures 124 can be placed around the circumference of the ring 123. The number and size of these apertures can be carefully selected so as to control the release rate of the high molecular weight water-soluble polymer, as well as other components with which it might be admixed, such as a low molecular weight water-soluble compound. Again, in a preferred embodiment, this combination of high molecular weight water-soluble polymer and low molecular weight water-soluble compound comprises the mixture of carrageenan and zinc salt discussed more fully in this application.

It is also within the scope of the present invention to create one-half of the IVR 123 including a groove 110 as shown in FIG. 15 containing the compressed high molecular weight water-soluble polymer therein. It is then possible to produce the second half of the IVR 123, again including a groove 110 therein as shown in FIG. 15, but in this case in which the groove is filled with the low molecular weight water-soluble compound admixed with the elastomeric polymers discussed above. In this embodiment, the two halves of the ring are then combined either by heat treatment or using a biocompatible adhesive or the like. In this embodiment, the aperture or apertures 124 can then be provided so as to contact both of the filled grooves containing both the high molecular weight water-soluble polymer and the low molecular weight water-soluble compound discussed herein.

It is also within the scope of the present invention to produce a groove 110, as shown in FIG. 15, either solely in the first half of the ring, or on both the first and second halves of the ring, in the manner discussed above. It is also contemplated, however, that the water soluble compound to be contained within the ring can be further encased within a polymeric sheath prior to completion of the ring in the manner discussed above. Thus, in this embodiment, the outer layer includes a pair of polymer layers, including the non-water-swellable elastomer of the ring itself and the polymeric sheath surrounding the inner layer. This embodiment is particularly adapted to be used where the overall IVR is intended to include a number of active agents comprising the water soluble and/or water insoluble molecules discussed herein, but preferably where they are incompatible with each other, and thus require physical separation. Thus, in this embodiment, the polymeric sheath, which can be either a water-swellable elastomer or a non-water-swellable elastomer, but preferably a non-water-swellable elastomer, and most preferably the same non-water-swellable elastomer as that of the ring itself, can include a water soluble and/or a water insoluble compound, such as those discussed herein. The polymeric sheath can, on the other hand, be free of any active agents.

In this embodiment, it is thus preferred that the outer layer comprising the non-water-swellable elastomeric polymers will include at least one and possibly additional water insoluble compounds. In a preferred embodiment, for example, this outer layer (the non-water-swellable elastomer) can include an NNRTI, preferably one such as MIV-150, either alone or combined with a contraceptive, such as levonorgestrel or the like. Furthermore, it is also contemplated that, in addition to the water soluble compounds of the inner layer, the inner layer can also include a water insoluble compound, such as those included in the outer layer of non-water-swellable elastomer.

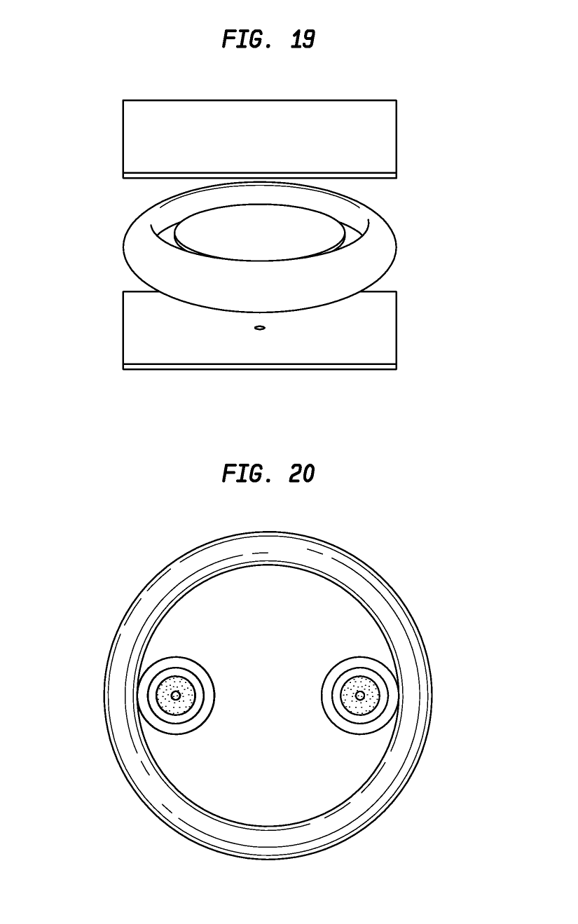

Another embodiment of the vaginal ring of this invention is shown in FIG. 18. In this embodiment, however, the ring 127 is a conventional ring, including a conventional sheath produced in the normal manner. In this case, however, a series of apertures or cavities 128 are cut or drilled into one of the surfaces of the ring 127, preferably to a substantial depth, but preferably not entirely through the ring 127. The number and size of these apertures can be selectively chosen, and each of these apertures can then be filled with pellets of compressed high molecular weight water-soluble polymer of the present invention either alone or combined with other high molecular weight water-soluble polymers, low molecular weight water-soluble compounds, and/or low molecular weight water-insoluble compounds. On the other hand, some of these apertures can be filled with pellets of low molecular weight water-soluble compounds of the present invention, combined with the elastomeric polymers discussed above, for sustained release therefrom. Again in this case, the release of these high molecular weight water-soluble polymers and/or the low molecular weight water-soluble compounds and/or other components contained in the compressed pellets can be controlled by the size of the apertures, the number of apertures, and the size of the pellets in each of the apertures.

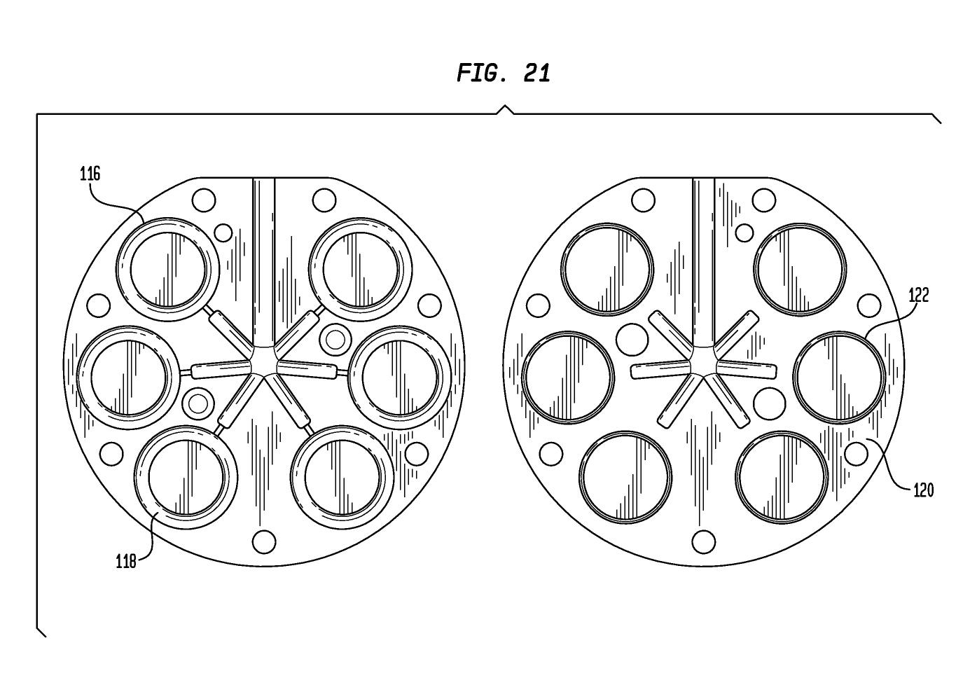

Yet another embodiment of the vaginal ring of the present invention is shown in FIGS. 19 and 20. In this case, once again a conventional ring of a small size (approximately 20 mm×2 mm) 130 is produced by conventional means. A pellet of compressed high molecular weight water-soluble polymer is then produced for placement in the center 132 of the ring 130. The compressed inner layer of high molecular weight water-soluble polymer preferably fits snuggly within the center 132 of the ring 130 and is retained therein. On the other hand, a pellet of low molecular weight water-soluble compound can be produced, again combined with an elastomeric polymer for enabling the sustained release of the low molecular weight water-soluble compound and then be placed in the center 132 of the ring 130. Once again, the inner layer of low molecular weight water-soluble compound is made to fit snuggly within the center 132 of the ring 130 and be retained therein. In order to then enclose the inner layer of water-soluble polymer within the outer layer, a pair of substantially planar sheets 134 and 136 of the non-water-swellable elastomer are affixed to the upper and lower surfaces of the ring 130, thus enclosing the pellet 134 therewithin. The materials of the substantially planar sheets (134, 136) can be the same material used to produce the ring 130 itself, or they can constitute membranes made from any other non-water-swellable elastomers that are non-reactive with the enclosed mixtures. To complete this portion of the device, an aperture 138 is drilled through at least one of the planar sheets, in this case sheet 136. This can include a single aperture 138 of a preferred size, or a number of apertures 138 of various or constant sizes for controlling release of the compressed high molecular weight water-soluble polymer therefrom. Referring to FIG. 20, the entire structure shown in FIG. 19 is duplicated so that two such structures (140, 142) with apertures 138 corresponding to the aperture shown in FIG. 19 shown therein. These devices (140, 142) are then contained within a larger or conventional-sized ring 146 (normally 55 to 60 mm×4 mm) so that they can be used for implantation as is the case with normal IVRs. The smaller rings can be affixed to the larger IVR by either using a bioadhesive or by physically creating hooks in the larger IVR, or injection molding a larger IVR into which the edge of the smaller IVR can be fixed. In this embodiment it is preferred that the IVR matrix itself for the large ring 146 also include low molecular weight water-insoluble compounds which are preferably compounded with the polymer of the ring and are dispersed therein for diffusion through the outer surface of the sheath of the ring 146.

Yet another aspect of the present invention is directed to a method for refining a non-absorbable, carrageenan. The formulation is typically prepared by mixing a solid buffer salt and lambda carrageenan, or the carrageenan mixture, in a weight ratio of from about 1:1 to about 10:1. The mixture of solid buffer salt and carrageenan is then solubilized in water or in an aqueous solution, to make the formulation. The pH of the formulation is then adjusted to be from about 6.0 to about 8.0. This is typically achieved by the addition of an acid, such as HCl or a base, such as NaOH. In general, the viscosity of the formulation is from about 20,000 to about 100,000 CPS, preferably from about 30,000 to about 35,000 CPS. At least one physiologically acceptable preservative can be added to the formulation. Examples of such preservatives are disclosed herein. The preservative can be present in the proportions indicated in the various pharmacopoeias, and in particular in a weight ration to the carrageenans of from about 80:1 to about 10:1, preferably from 40:1 to about 15:1.

Solid buffer salts include solid alkaline metal salts of acetic acid, citric acid, phosphoric acid, and lactic acid. In the case of phosphoric acid, the solid alkaline metal phosphate buffer includes solid mixture of tri-basic and di-basic alkali salts of phosphate, preferably in anhydrous form, wherein alkaline metal includes, but is not limited to, potassium and sodium. Any physiologically acceptable buffer can be used. However, in the case where water-soluble zinc salts are utilized, the phosphates are less preferred, and in these formulations, the acetates, citrates and lactates are more preferred. In preferred embodiments, these buffer solutions comprise mixtures of acetic acid and sodium acetate; citric acid and sodium citrate; and lactic acid and sodium lactate.

Without intending to be bound by any particular theory of operation, it is believed that the carrageenans are dry powders that are extremely hygroscopic when exposed to the atmosphere. The uptake of atmospheric moisture into the dry ingredient causes clumping of the material. The problem compounded when the material is then introduced into the aqueous base solution, such that complete incorporation of the carrageenans into a homogeneous aqueous solution cannot be obtained. It is also believed that by mixing the carrageenans and at least one solid buffer salt together, the solid buffer salt absorbs the atmospheric moisture that the carrageenans would have absorbed when exposed to the atmosphere, thus preventing or substantially reducing clumping of the carrageenans. It is further believed that the process serves to increase the soh ility of carrageenans in water, and achieves stabilization of the pH.

The following examples are intended to further illustrate certain embodiments of the invention and are not intended to limit the invention in any way.

### Example 1

### Production of 500 Liters of the Carrageenans

In preparing the lambda carrageenan or the carrageenan mixture, (1) the formulation ingredients should be weighed individually in a clean, dry weighing vessel; (2) the ingredient's “actual” weight, not protocol weight, should be recorded in the manufacturing production log regardless of even slight variation between the two; (3) any bulk ingredient container containing an artifact(s) or contaminate should not be used and the container should be closed, sealed, marked “CONTAMINATED” and removed from production area; (4) in process production batch should not be transferred from one vessel to another before manufacturing is completed and formulation has passed quality control testing; and (5) production vessel should remain closed during manufiicturing to avoid loss of water due to evaporation, especially during any steps that require heating.

Additionally, carrageenan has proven to be stable in the solid state and the production state under a variety of adverse conditions, including freezing or autoclaving, for 24 months.

The following pertains to a procedure for that was used to make a formulation containing a carrageenan mixture of lambda (λ) and kappa-II (K-II) carrageenans (the (K-II/λ carrageenan mixture). In the course of preparing the K-II/λ carrageenan mixture from 100 mL laboratory size batches on to scale-up of 15 and 30 liter laboratory batches to finalizing the manufacturing procedure of 500 liter batches, it became difficult to obtain batch-to-batch consistency of the desired formulation. The present method surprisingly overcame these difficulties and produced formulations of the K-II/λ carrageenan mixtures having consistent batch-to-batch quality

Equipment:

Production Vessel—IKA, EMA 9/500AIUTL is a water jacket production vessel that allows for rapid heating and cooling of solution during production.

Ingredients:

the K-II/λ carrageenan mixture;

Phosphate buffer saline (PBS) [containing: NaCl—120 mmol/L, KCl—2.7 mmol/L Phosphate buffer (potassium phosphate monobasic and sodium phosphate dibasic)—10 mmol/L (Sigma Aldrich, Saint Louis Mo.);
p-Hydroxybenzoic methyl ester (Methyl paraben)—(Nipa Laboratories, Pontypridd, UK);

Hydrochloric Acid (HCl)—Merck, Damistadt, Germany;

Purified water—Clean Chemical Sweden AB, Borlange, Sweden.

### Procedure

(1). Weighed ingredients in the following quantities:

| INGREDIENT | QUANTITY |
|---|---|
| Purified water (3 Parts) | 484.0 kg |
| the K-II/λ carrageenan mixture | 15.0 kg |
| Phosphate buffer saline (PBS) | 4.8 kg |
| Methyl paraben | 0.5 kg |
| Hydrochloric acid (10%) | 0.5 kg |

(2). Carefully and thoroughly mixed the dry ingredients, the K/λ carrageenan mixture and Phosphate buffer saline (PBS) together;

(3). Inspected production vessel to ensure that mixing chamber is clean, dry and free of artifacts, and that the bottom value is closed;

(4). Filled the production vessel with 100.0 L (Part I) of purified water and began stirring:

turbin 500 rpm and anchor 20 rpm. Water is added in 3 parts. The first part was enough to dissolve the methyl paraben. The second part aided in reducing the temperature, sufficiently diluted the HCl so acidic hydrolysis of carrageenan did not occur while maintaining low enough solution level so when adding the carrageenan/PBS mixture, the delivery sieve could be lowered into the mixing vessel such that it did not come into contact with the base solution and was lower than the vessel access hatch so the excessive ‘dusting’ of the mixture was not lost. The third part completed the final concentration.

(5). Continued stirring and add 0.5 kg of methyl paraben and 0.5 kg of HCl. Closed vessel access hatch and heat water 750 to 850 C. Once this temperature was reached, we continued stifling for a minimum of 10 minutes to dissolve methyl paraben.

(6). Discontinued heating and add 250.0 kg (Part II) of purified water. Cooled solution to 250 to 300 C. The addition of the water expedited the cooling process. The solution needed to be cooled so that it was not producing steam when the next addition of ingredients was made. Besides preventing water loss when the vessel was open for the next addition, steam caused the carrageenan/PBS: mixture to clump and stick to the sieve that was used in the addition:

(7). Opened access hatch and began the addition of carrageenan/PBS mixture slowly through a sieve with gentle shaking. Addition took approximately 20 minutes. Coincided the addition of the mixture with increasing the stirring speed to a maximum speed of turbin 1200 rpm and anchor 20 rpm. The viscosity of the solution increased exponentially with the addition of the carrageenans. If the stirring speed was not significant, the carrageenan formed ‘hydro-sealed’ clumps, which never became dissolved and incorporated into the solution, thereby rendering the batch unacceptable. (‘Hydro-sealed’ clumps are pockets of dry carrageenan, which are surrounded with an outside coating of semi-hydrated carrageenan, which become impenetrable to water due to carrageenan's extremely large molecular weight and flexible structure.);

(8). Closed access hatch and continued stirring at maximum speed, turbin 1200 rpm and anchor 20 rpm. Added 134.0 kg purified water (Part III) and disconnect the waterline, close value. Heated solution to 750 to 800 C by applying 52% heat; and

(9). Checked that all the values were closed and applied the vacuum to the vessel at 400 mbar. Stirred solution at slightly reduced speed, turbin 1100 rpm and anchor 20 rpm, under vacuum for 1.5 hr at 750 to 800 C. The constant stirring of the solution, which was necessary for even distribution and complete incorporation of ingredients, caused excessive air entrapment. The vacuum pulled this air out of the solution;

(10). Turned heating OFF, stirring OFF, and vacuum OFF. Removed Testing Sample from production vessel and tested for Control Test #1 Completed incorporation and even distribution;

Control Test #1: Complete Incorporation and Even Distribution

Removed approximately 90 μL of the in-process mixture (used a large orifice 200 μL pipette tip to aid in removing the carrageenan solution) and mixed in 10 μL of a 0.1% methyl blue TS (1:1, isopropyl alcohol: dH2O) in a 500 μL Eppendorff tube. The mixture in the tube should appear as an even blue color. This indicates that the K-II/λ carrageenan mixture is evenly distributed within the solution. Prepared a microscope slide with a 10 μL of this mixture; covered with a cover slip and viewed under low magnification (10×). The K-carrageenan mixture should appear as large purple strands. This indicates that the K-II/λ carrageenan mixture was completely incorporated and the solution is “PASS”. If the strands are blue or large blue chimps are visible, then the K-II/λ carrageenan mixture is not completely incorporated and solution is “FAIL.”. Continued processing the solution under the conditions of step #9. Rechecked solution at 0.5 hour intervals until solution is “PASS”.

(11). When the solution is “PASS” for Control Test #1, test for Control Test 42, pH;

Control Test #2: pH

The testing sample should be cooled to 2.5° C.± 2 (a range of 23° C. to 27° C.) for testing. The pH should be 7.0±0.1 (a range of 6.9 to 7.1). This indicates that the solution's pH is uniform and the solution is “PASS”. If the solution is not within the acceptable pH range (6.9 to 7.1) the solution is “FAIL”. If the solution is “FAIL”, the solution needs to be adjusted, as needed with either 10% HCl (to decrease the pH) or 1N NaOH (to increase the pH) in 25 ml, increments until the solution is “PASS”. With each incremental addition of either acid or base, thorough stifling (stirring and vacuum condition step #9, no added heat) is needed to ensure even distribution throughout batch before re-testing the pH. Recheck solution after stirring/vacuum for 0.5 hour. Continue in this manor until solution is “PASS”.

(12). When the solution is “PASS” for Control Test #2, begin cooling the mixture to 25° C.

±2° (2.3° C. to 27° C.). The stirring speed, which should be OFF at this point, will need to be increased as the solution thickens upon cooling. At start, turbin OFF and anchor 20 rpm, increase turbin 20 rpm/15 min and increase anchor 10 rpm 30 mm, ending with turbin 1000 rpm and anchor 40 rpm. It is preferred not to increase stirring to rapidly; otherwise, air entrapment may result. If this should happen, apply the vacuum 400 mbar until solution is free of air bubbles:

(13). Remove Final Testing Sample from the production vessel and retest for Control Test #2 pH and for Control Test #3, Viscosity.

Control Test #3: Viscosity

The testing sample should be heated to 35° C.±2″ (a range of 33° C. to 37° C.). To optimize performance, the viscosity should be about 30,000 to about 40,000 cP. Viscosity measurements indicate, that the solution's viscosity is uniform with the PC Reference sample and CCS production batches and the solution is ‘PASS”. If the solution is “RAIL” obtain testing samples from the top and the bottom of production vessel and conduct Control Test pH and Control Test #3, Viscosity on each sample. If the solution is still “FAIL”, repeat step #9 and step #12 and retest the solution for Control Test 3#, Viscosity. If solution is “FAIL” an Out of Specifications Study shall be undertaken to determine the source of out of specification production.

It was discovered that adjusting viscosity with the addition of water yields an unknown percent/concentration to the final production batch rendering the production batch unacceptable.

(14). When the solution is “PASS” for Control Tests #1, #2, and #3 it is an acceptable production’ batch which can be processed for the final control testing. Connect the transfer tube containing a filter bag to the bottom value of the production vessel and transfer the formulation into storage containers. Retain a Test Sample for Microbiological Testing before filling applicators.

The final formulation prepared in the process discussed above has the following components.

Weight/Percent: 500 Liters of formulation

| | | |
|---|---|---|
| Component | Weight | Percent |

| | | | |
|---|---|---|---|
| Purified Water | 484.0 | kg | 96.8 |
| Methyl paraben | 500 | g | 0.1 |
| PBS: | | | |
| NaCl | 120 | mmol/L | |
| KCl | 2.7 | mmol/L | |
| Phosphate salts | 10 | mmol/L | |
| 10% HCl | 500 | g | 0.1 |
| the K-II/λ carrageenan | 15 | kg | 3.0 |
| mixture | | | |

The final formulation has a pH of about 7.0 which was adjusted by adding. HCl solution and 1:1 ratio of K3PO4 and Na2HPO4.

### Example 2

### Effect of Carrageenan on HIV Infections In Vitro

Carrageenan has been shown to block HIV and other enveloped viruses by several laboratories including the laboratory of the P115-19. Several different types of target cells and strains of HIV have been employed in these studies. Generally, 50% blocking is observed at a few micrograms/mL. This result is similar to other sulfated polysaccharides such as heparin and dextran sulfite.

### Example 3

### Intra-Vaginal Viral Infection Studies—HSV-2/Mouse

The HSV-2/mouse (Balb/C) system is widely utilized by most investigatory groups engaged in the development of a microbicide. An important difference between the system established by Phillips 20-22 and other systems is the utilization of viral dose range comparison. The standard viral challenge dose, 100% infection dose or 104 pfu, used by others for evaluation of a microbicide is rate limiting. The large majority of the microbicides under development., as well as many of the OTC spermicides will show a significant rate of protection against HSV-2 infection at this viral challenge doses. However, Phillips has utilized a virus concentration method that will enable evaluation at viral challenge doses of 105, 106, and 1,000×100% infection dose.

Using this viral challenge dose system, a comparison study was conducted to evaluate the comparison protection rates of a number of microbicides under development, OTC spermicides and lubricants, and possible formulations for use as a placebo in the clinical trials to evaluate efficacy of a microbicide. In addition to a composition of the present invention containing the K-II/λ carrageenan mixture (also referred to herein as the “K/λ carrageenan composition”), comparative test formulations were: microbicides under development such as BufferGel™ and No Fertil, OTC spermicides: K-Y Plus® Gynol II®, and Advantage STM; OTC vaginal lubricants: Replens® and K-Y Jelly®; and possible placebo formulations: 2.5% Carbopol® and 2.5% methyl cellulose.

Test formulations fell into three categories with respect to efficacy in protecting mice from vaginal HSV-2 infection. At the viral challenge dose of 104 pfu, with the exception of K-Y Jelly, Carbopol and methyl cellulose, all formulations provided a significant level of protection against infection from HSV-2. However, at the viral challenge dose of 105, with the exception of the K-II/λ carrageenan composition, all formulations only provided a minimum level of protection. The K-II/λ carrageenan composition was the only formulation still affording a level of protection against viral infection at the viral challenge dose of 106 pfu 20 By evaluating various formulations in the viral dose range comparison system the resulting data was the first demonstration of the unexpected high level of protection against viral infection that the K-II/λ composition provides.

Therefore, it can be concluded that the HSV-2/mouse system can be employed as a means by which candidate microbicides can be evaluated and compared under the same testing conditions to identify potential effective microbicides.

### Example 4

### Duration of Activity—HSV-2/Mouse

One of the criteria, set forth by UNAIDS (World Health Organization, AIDS branch) for an ideal microbicide states it should be active upon insertion and for a long period of time, giving a woman more flexibility in product use. Additionally, the time course for infection by cell-free or cell-associated HIV to occur may not be immediate. The HSV-2/mouse system can be employed to evaluate the duration of time that a microbicide would retain activity. This is done by intra-vaginal application of a test formulation, waiting a set period of time, and then challenging mice with a known dose of virus. “Duration of activity” testing was conducted using Gynol HS (a 2% N-9 containing OTC spermicide), BufferGel® (a low pH microbicide under development) and the K-II/λ carrageenan composition, at five minutes and 1.5, 3, 6 and 18 hours following formulation application. By the 1½-hour time point, Gynol II® no longer afforded any protection against infection and BufferGel® had dropped to being only 30% effective. BufferGel's efficacy continued to drop over time and no longer afforded any protection by 6 hours. In marked contrast, the K-II/λ carrageenan composition remained 85-100% effective in protecting against HSV-2 infection up to 6 hrs and remained 72% effective at 18 his. The K-II/λ carrageenan composition continued to retain some level of activity for up to 24 hours. See FIG. 1. The extended duration of protection from viral infection is unique to carrageenan, in particular K-II/λ carrageenan composition.

### Example 5

### Intra-Rectal Viral Infection Studies HSV-2/Mouse

Ideally, a microbicide that was effective in protecting against infection by HIV could be used rectally as well as vaginally. Using an intra-rectal viral challenge modification of the HSV-2-/mouse system an evaluation of the efficacy and safety of a microbicide was explored.

Pre-treatment of the rectum with the K-II/λ carrageenan composition significantly reduced the number of animals that became infected following rectal challenge with HSV-2, compared to pretreatment with PBS or methylcellulose (an inert placebo)23

### Example 6

### Effect of Carrageenan Composition on Vaginal Flora

It is important that the use of a microbicide does not disrupt the balance of the natural vaginal flora. In vitro studies indicated that carrageenan did not enhance or inhibit the growth rate of Lactobacillus acidophilus, the most common bacterium present in the vaginal flora. A study conducted in 35 women participating in a Phase I clinical trial for the vaginal safety of the K-II/λ carrageenan composition showed no significant change in vaginal flora, as measured by the presence or absence of bacterial vaginosis 13.

### Example 7

### HIV/Mouse Viral Transport System

Although mice can not be infected with HIV, it has been shown that when active or inactivated virus is instilled into the vagina of mice, virus can be subsequently detected in the lymph lodes by the use of reverse transcriptase polymerase chain reaction (RT-PCR)24 Evidence has been presented that dendritic cells played a role in the uptake of virus and subsequent transport to the lymph nodes. This conclusion is in agreement with studies implicating dendritic cells in the initial stage of sexual transmission of HIV25.

Results indicate that the K-II/λ carrageenan composition is efficacious in preventing HIV from reaching the lymph node, presumably by blocking HIV transport from the vagina via dendritic cells.

HIV transport using a mouse system and AldritholTM-2 inactivated virus were used. This is a standard method for inactivating HIV that does not alter the viral envelope. The spleen and the lymph nodes were assayed for the detection of HIV in order to establish the spleen as an alternate repository site for HIV. The spleen (as opposed to the lymph nodes) allows for obtaining relatively larger amounts of RNA for performing RT-PCR for the detection of HIV. In addition, extraction of spleens is less time consuming, than removal of the lymph nodes thereby lessening the probability of RNA degradation.

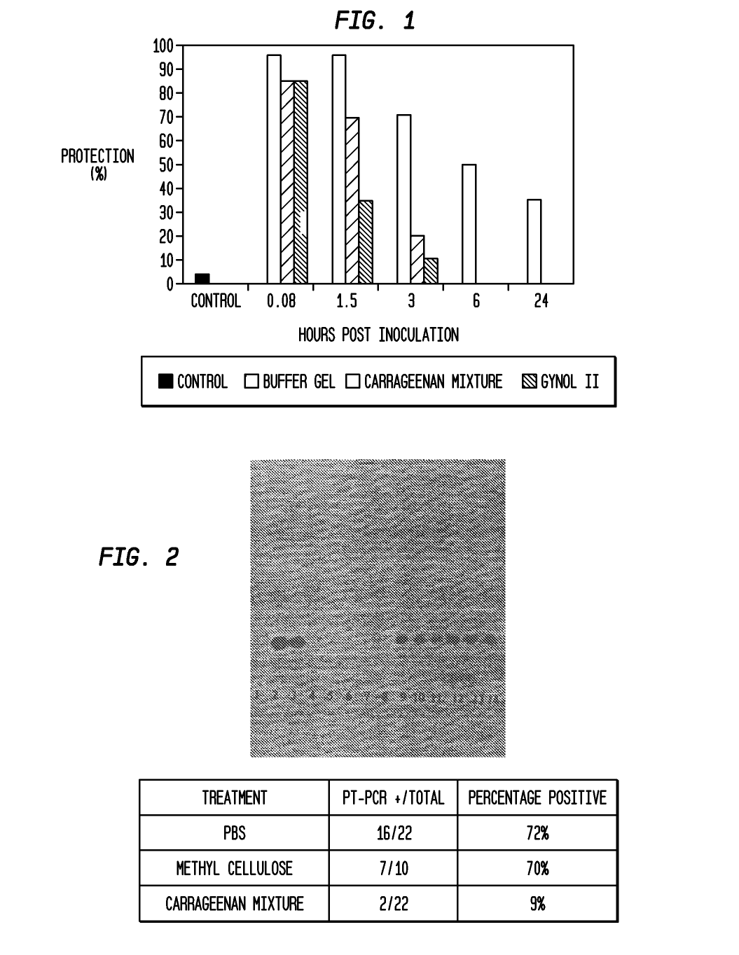

To determine the efficacy of the K-II/λ carrageenan composition in preventing HIV from crossing the cervical/vaginal barrier, mice were randomized into three groups: 1) non-treated PBS control mice; 2) mice pre-treated with methyl cellulose (inert placebo); and 3) mice pretreated with the K-II/λ carrageenan composition. Results are shown on the Southern Blot in FIG. 2 and the table below.

| | | Percentage Positive |
|---|---|---|
| Treatment | PT-PCR+/total | (Infected) |
| PB5 | 16/22 | 72% |
| Methyl Cellulose | 7/10 | 70% |
| K-II/λ carrageenan | 2/22 | 9% |
| composition | | |

Data from PBS (control) and methyl cellulose treated and mice treated with the K-II/λ carrageenan composition show that the K-II/λ carrageenan composition significantly reduced the number of positive (i.e., infected) animals, and that methyl cellulose had no effect as compared to PBS (control). The data also indicate that the K-II/λ carrageenan composition was effective in preventing HIV from leaving the vaginal vault.

### Example 8

### Cell Trafficking/Mouse System

It has previously been suggested that sexual transmission of HIV could be mediated by HIV-infected lymphocytes or macrophages in semen that cross the genital tract epithelium26,27. In order to test the hypothesis that mononuclear blood cells traffic from the vaginal vault through intact epithelia, double-vitally-stained activated mononuclear blood cells (mouse) were placed in the vagina of mice. Four hours later, animals were sacrificed and iliac and inguinal lymph lodes and the spleen were removed and cells were dissociated and count by fluorescence microscopy. Numerous double-stained cells were observed in the iliac and inguinal lymph nodes and the spleen28, XX. To evaluate the effect that the carrageenan composition may have on blocking this process, animals were pre-treated with the test formulation prior to instillation of labeled cells.

| | | Inguinal & Iliac | |
|---|---|---|---|
| Mouse | Inoculation | Lymph nodes | Spleen |
| 1 | Macrophages | 36 | 555 |
| 2 | Macrophages | 52 | 366 |
| 3 | Macrophages | 59 | 672 |
| 4 | Macrophages | 87 | 786 |
| 5 | Macrophages | 61 | 357 |
| 6 | Macrophages | 40 | 859 |
| 7 | Macrophages | 54 | 312 |
| 8 | K-II/λ carrageenan + | 4 | 30 |
| | Macrophages | | |
| 9 | K-II/λ carrageenan + | 4 | 6 |
| | Macrophages | | |
| 10 | K-II/λ carrageenan + | 6 | 48 |
| | Macrophages | | |
| 11 | K-II/λ carrageenan + | 3 | 53 |
| | Macrophages | | |
| 12 | K-II/λ carrageenan + | 3 | 3 |
| | Macrophages | | |
| 13 | Methyl cellulose + | 14 | 120 |
| | Macrophages | | |
| 14 | Methyl cellulose + | 27 | 245 |
| | Macrophages | | |
| 15 | Methyl cellulose + | 38 | 96 |
| | Macrophages | | |

Donor's cells were present both in the iliac and inguinal lymph nodes and in the spleen. When mice received only a vaginal inoculation of macrophages, the recipient animals had an average of 55 labeled donor's cells in the draining lymph nodes and of 558 cells in the spleen, respectively. In mice that received a vaginal pre-inoculation of K-II/λ carrageenan composition (indicated in table above as “K-II/λ carrageenan”) an average of only 4 cells were counted in the draining lymph nodes, and an average of only 28 were observed in the spleen. The difference between untreated and K-II/λ carrageenan composition-treated animals was significant. When the recipients were pre-inoculated with methyl cellulose, the number of donor's cells that reached lymph nodes and spleen averaged 26 in the lymph nodes and 153 in the spleen. The difference between K-II/λ carrageenan composition-treated mice and methyl cellulose-treated mice was significant, whereas the difference between untreated mice and methyl cellulose pre-inoculated mice was not significant. No fluorescent cells were observed in control mice that had been inoculated with frozen-thawed CMTMR stained macrophages.

### Example 9

### Microbicide Effect on Papillomavirus

The K-II/λ carrageenan composition has also been proven effective on blocking bovine papillomavirus (BPV) foci formation in vitro (data not shown). The carrageenan composition is efficacious in preventing human papillomavirus (HPV) from transforming human vaginal explants in a xenograft system. The SKID mouse xenograph system employs explants of human vaginal tissue rolled into cylindrical tubes that are grafted subcutaneously on NOD/SKID (immunodeficient) mice29. The grafts are allowed to heal for two weeks, at which time one end of the tube is opened and a test compound is instilled followed by HPV challenge. In experiments evaluating the K-II/λ carrageenan composition, in 14 out of 14 saline treated control explants were transformed. In contrast, only 1 out of 17 explants treated with the K-II/λ carrageenan composition was transformed (data not shown).

### Example 10

### Effects of the K/λ Carrageenan Mixture in Dilution Assay

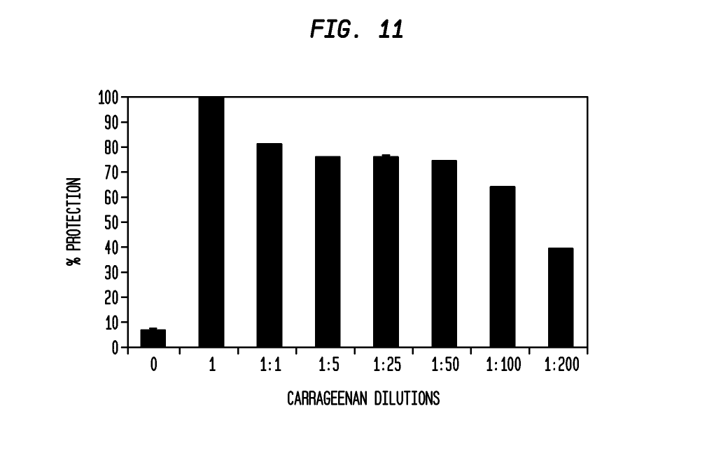

The K/λ carrageenan mixture is also effective at high dilutions as demonstrated in the HSV-2 mouse system. A 3K-II/λ carrageenan composition was diluted in PBS to make 1:1, 1:5, 1:25, 1:50, 1:100, and 1:200 dilutions. Dilute solutions were vaginal administered to mice followed by 104 (100% infection dose) of HSV-2. The results from these experiments are unexpected. Instead of observing a dose dependent decrease in the anti-viral protection rate the K-II/λ carrageenan composition dilution of 1:50 retained most of the anti-viral protection rate as less dilute solutions. Furthermore, significant activity was retained even with the 1:200 solution. See FIG. 11.

### Example 11

### Effects of the K-II/λ Carrageenan Composition-Based Formulations Against HIV

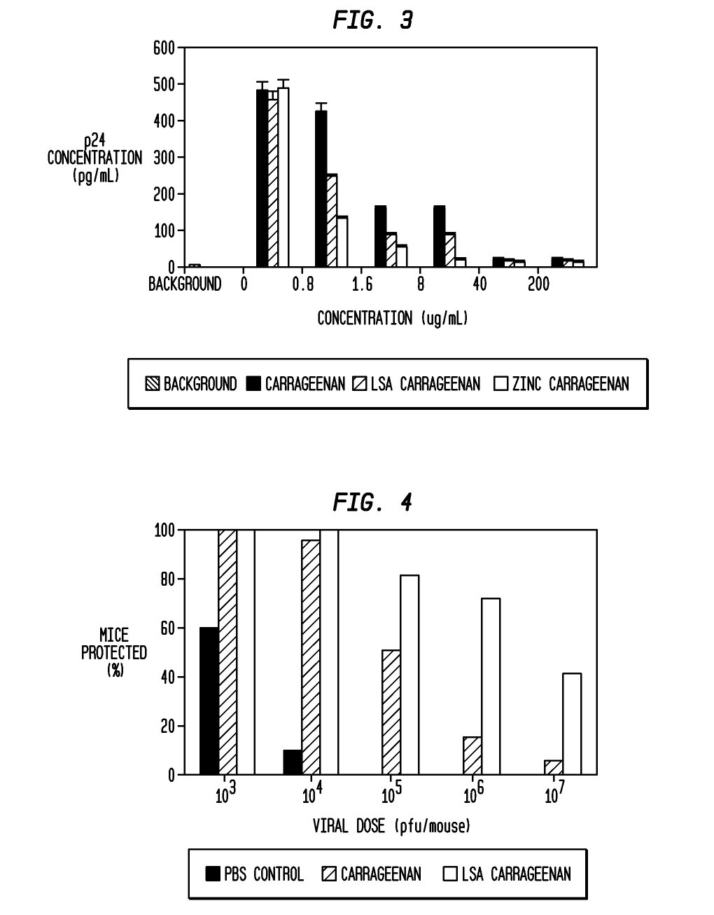

Compounds have been identified which when added to, or bound to the carrageenans of the present invention, significantly increase efficacy in blocking HIV infection of PBMCs in vitro. Studies on the effectiveness of Zn-carrageenan and LSA-carrageenan on blocking HIV infection of PBMCs have shown that both formulations are more effective than a compositions containing the carrageenans alone at lower concentrations. The testing results are shown in FIG. 3.

### Example 12

### Effects of LSA-Carrageenan Against HSV-2

Results indicate that LSA-carrageenan is more efficacious in blocking HIV infection than the carrageenans. (See FIG. 4.) Originally LSA did not seem to be an ideal candidate compound for a microbicide due to the fact of its brown coloration. However, it was found that a concentration of 0.25%, LSA is highly effective and imparts negligible coloration when formulated. In order to ensure that LSA would not impart discoloration, white cotton fabric was soaked overnight in 3% LSA and then rinsed with tap water; the results revealed no change in the color of the fabric. LSA-carrageenan was compared to carrageenan in the HSV-2/mouse system in order to determine efficacy in blocking viral infection in vivo. Preliminary results showed that LSA-carrageenan was more efficacious than carrageenan in blocking viral infection.

In addition to the results presented above. LSA-carrageenan was compared to the K-II/λ carrageenan composition alone at a viral challenge dose of 106 pfu, in three separate experiments. LSA-carrageenan was significantly more effective than the K-II/λ carrageenan composition alone in all experiments. The addition of other sulfated polymers to K-II/λ carrageenan composition did not increase the effectiveness of the formulation. For example, the addition of 5% dextran sulfate or 5% heparin to K-II/λ carrageenan composition had no effect on efficacy against HSV-2 infection in mice.

Evaluation of K-II/λ Carrageenan Composition (Referred to in the Three Tables Below as “Carrageenan”) Formulations with and without LSA

HSV-2 106 pfu viral dose is equivalent to 100 times the viral dose that would infect all unprotected mice. It is necessary to use such high closes of virus because carrageenan is extremely effective at inhibiting viral infection.

Each formulation is initially tested in a total of 20 mice. Compounds or formulations that show a blocking effect are assayed again in another 20 mice. The number of mice infected is an average.

| | # MICE INFECTED | |
|---|---|---|
| FORMULATION | TOTAL # MICE | % INFECTED |
| 3% Carrageenan | 14/20 | 70 |
| 1% Carrageenan | 20/20 | 100 |
| 0.5% Carrageenan | 20/20 | 100 |
| 3% Carrageenan + 3% LSA | 4/20 | 20 |
| 3% Carrageenan + 1% LSA | 2/20 | 10 |
| 3% Carrageenan + 0.5% LSA | 4/20 | 20 |
| 3% Carrageenan + 0.25% LSA | 5/20 | 25 |
| 3% Carrageenan + 0.1% LSA | 7/20 | 35 |

The viral dose is 100 times the 100% infection rate and no compound other than the minimal effect of 3% carrageenan has had any effect at such a high virus dose.

Subsequently, LSA was assayed without Carrageenan to better evaluate its inhibitory properties. LSA was added to the inert thickener, methylcellulose, to maintain the same viscosity that vaginal products (lubricants, spermicides, and microbicides) generally have. (Data shown below.)

Evaluation of LSA without Carrageenan

| | # MICE INFECTED | |
|---|---|---|
| FORMULATION | TOTAL # MICE | % INFECTED |
| 3% Carrageenan | 14/20 | 70 |
| 3% LSA - methylcellulose | 8/20 | 40 |
| 1% LSA - methylcellulose | 8/20 | 40 |

LSA proved to be more effective than carrageenan, showing better blocking of HSV-2 infection than carrageenan. However, the combination of the two ingredients out-performed either one alone.

### Example 13

### Use of LSA in Microbicides

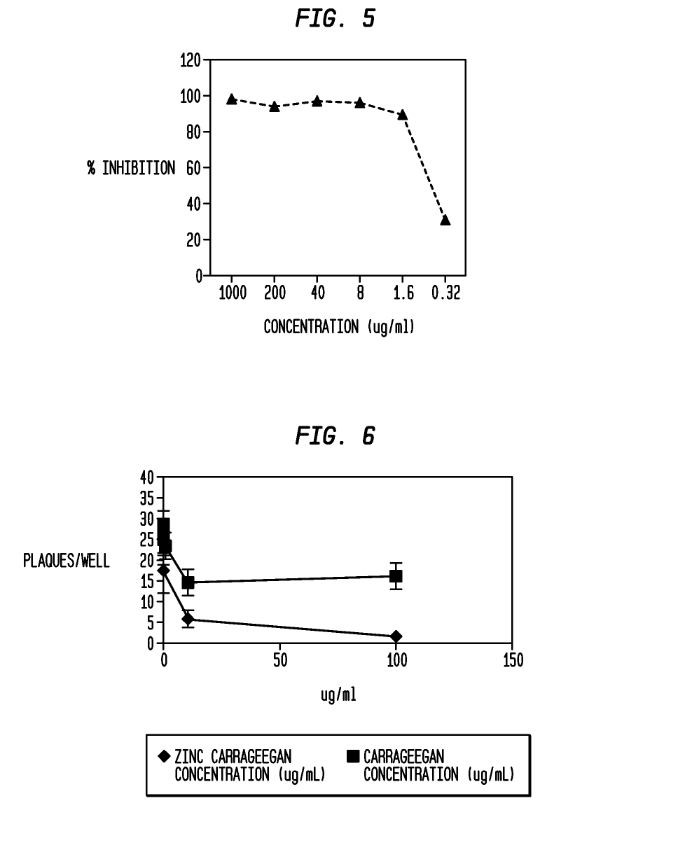

LSA is effective as a microbicide against HSV-2 infection, HIV and other STI's, with or without carrageenan. The sulfated polymer LSA is effective in protecting epithelial cells in vitro against HIV infection and mice from HSV-2 infection. The inhibitory effect may be observed with other enveloped viruses such as the human pathogen, human T cell leukemia virus. In addition, epithelial cells are protected against the human papillomavirus, which is not an enveloped virus. The inhibitory efficaciousness of LSA may thus extend to a broader range of STI's. The testing results are shown in FIG. 5.

### Example 14

### Effects of Zn-Carrageenan Against HSV-2

Studies on the effectiveness of Zn-carrageenan against HSV-2 infection have been conducted in vitro and in vivo. In vitro studies assayed the effect of Zn salts alone in preventing plaque formation in the HSV-2 plaque assay41. Zn salts were found to have an IC50 at a 50 mM concentration in reducing plaque formation. It was observed that Zn-carrageenan is significantly more effective than carrageenan or Zn salts alone in preventing plaque formation IC50<10 μg/mL, or <25 mM. The testing results are shown in FIG. 6.

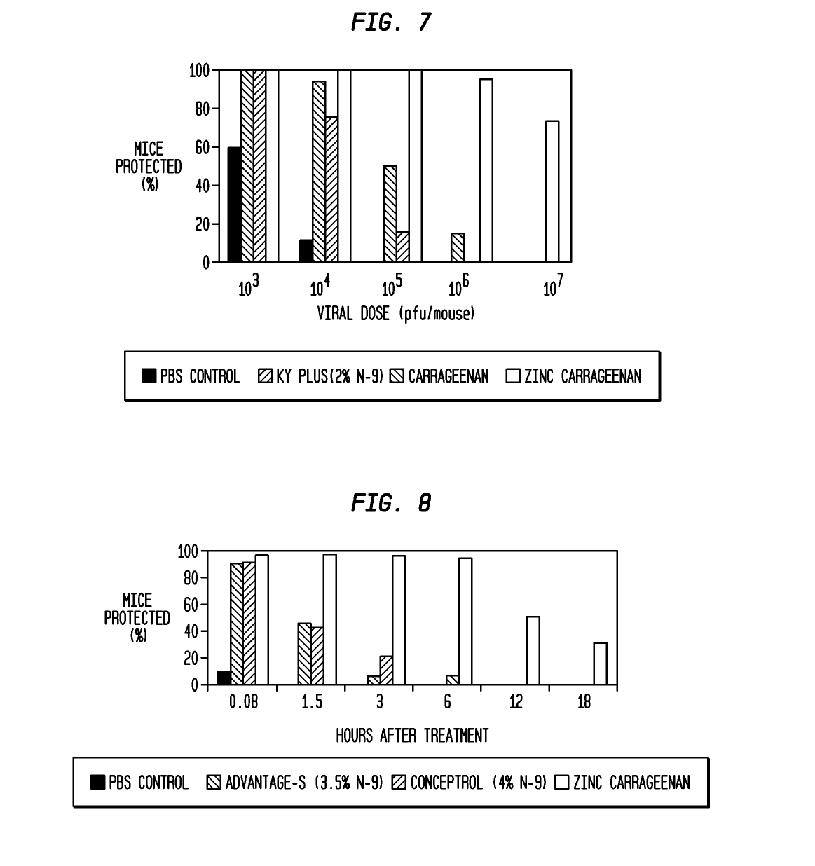

Zn-carrageenan has also been evaluated in the HSV-2/mouse system (see FIG. 7). In order to compare Zn-carrageenan with the OTC spermicide K-Y Plus and the K-carrageenan composition, HSV-2 viral challenge doses ranging from 103 pfu or 50% infection dose, to 107 pfu or 1000×100% infection dose was also used. Applicants had determined that K-II/λ carrageenan composition could protect some animals at a viral challenge dose of 106 pfu or 100×100% infection dose. No other candidate microbicide tested was able to afford protection at this viral dose. In preliminary studies it has been observed that Zn-carrageenan significantly protect mice against HSV-2 infection at this dose as well as at a viral challenge dose of 107 or 1,000×100% infection dose. The fact that the addition of Zn to the K-II/λ carrageenan composition (to form a complex) increased the level of anti-viral protection was most unexpected.

### Example 15

### Zn-Carrageenan Duration of Activity

The K-II/λ carrageenan composition remains active in the mouse vagina for an extended period of time. Similar experiments were carried out to compare Zn-carrageenan to two OTC spermicides, Advantage S and Conceptrol for duration of activity. It was observed that Zn-carrageenan did not lose any level of activity in 6 hours, where Advantage S and Conceptrol showed a 50% reduction in activity at 1.5 hours and by 3 hours were no longer able to afford protection (see FIG. 8).

### Example 16

### Zn-Carrageenan Efficacy Post-Viral Challenge

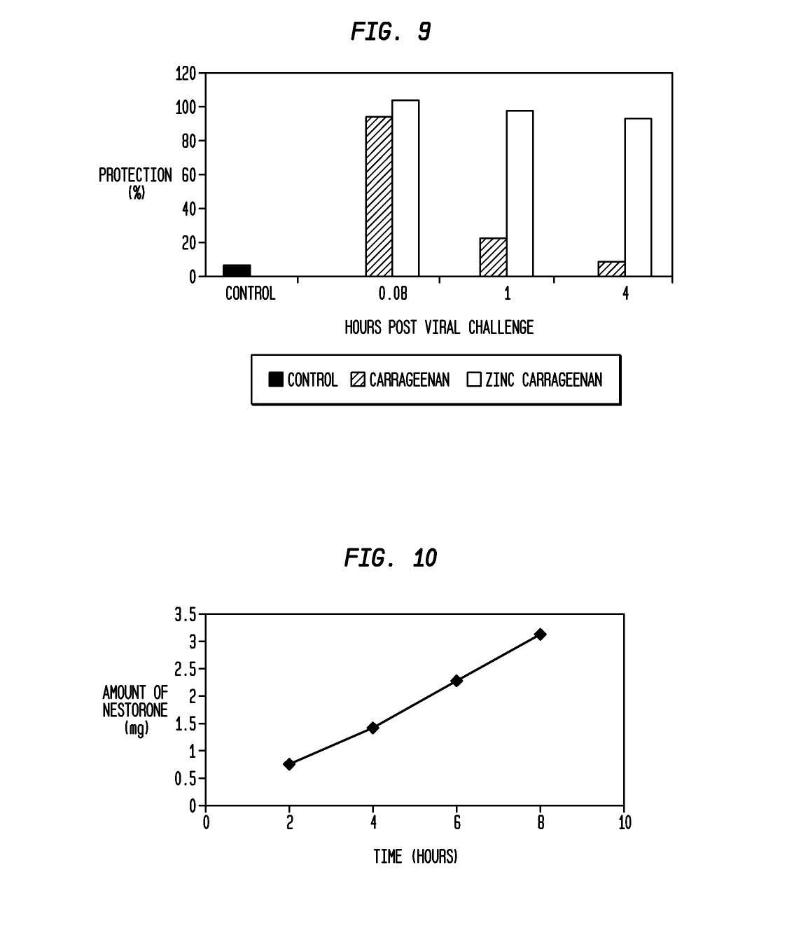

A microbicide that was able to be effective even if administered following exposure to a virus would extend product use to include women who were not able to use the product until after intercourse had already occurred e.g., women who fell victim to rape. Previously, researchers have been unable to identify a microbicide that might afford such protection. Zn-carrageenan is able to afford protection against HSV-2 infection in mice post-viral challenge. As the data below demonstrate, Zn-carrageenan is exceptional in that it demonstrated activity for up to 4 hours post-viral exposure (see FIG. 9). This finding is remarkable in light of Applicants' observations that K-II/λ carrageenan composition did not prevent infection post viral challenge unless administered immediately following HSV-2 challenge.

### Example 17

### Contraceptive Microbicide for Dual Protection

The K-II/λ carrageenan composition remains in the vagina for up to 24 hours, enabling a once-daily application for protection against HIV and its use as a vaginal delivery system for a contraceptive hormone. The feasibility of delivering various steroids vaginally has been thoroughly investigated with the recent development of contraceptive vaginal rings43. It has been shown that steroids applied directly to the vaginal mucosa are quickly absorbed, and only very small doses are needed to achieve the desired contraceptive effect 48-52. In addition, vaginal delivery is usually accompanied by diminished undesirable side effects that are often associated with oral contraceptives.

The vaginal formulations of the present invention provide dual protection as a combination microbicide/contraceptive that have a further advantage of enhancing user motivation for compliance. The contraceptive hormone NES is a preferred contraceptive agent. This synthetic progestin has been shown to be an exceptionally potent molecule. Using classic bioassays of measuring the progestational potency. NES has proven to be 100 times more active than progesterone and only very small quantities of NES are required to suppress luteal activity. Additionally, extensive toxicology studies of NES have been conducted.

### Example 18

### Diffusion of NES from the K/λ Carrageenan Mixture

In order for the formulation containing the K-II/λ carrageenan composition and NES (hereinafter “CARRA/NES”) to be an effective contraceptive, it is essential that NES be released from the carrageenan and absorbed through the vagina. We have carried out in vitro assays to determine if NES is released from CARRA/NES.

We examined diffusion of NES through a dialysis membrane with a molecular weight cutoff of 1000. The molecular weight of NES is 370. NES diffused from the dialysis bag at a constant rate, as measured by HPLC. Results are illustrated in FIG. 10. These results demonstrate that NES is not bound to carrageenan. However, the rate of diffusion observed through the dialysis membrane cannot be related to the rate of diffusion that would be observed in the human vagina as the rate of diffusion was dependant on the surface area of the dialysis bag. Conditions in the vagina would be different.

We also conducted an experiment that involved centrifuging CARRA/NES through an Ultrafree-15 centrifugal filter and tube assembly at 2000 g for 99 minutes, to calculate percentage of NES released. The centrifuge filter is a device that fits into a centrifuge tube. The device has a flouted filter in the bottom that allows molecules with MW under 500 to pass through. Using this device, over 98% of the added NES was recovered in filtrate. This experiment confirms that NES is not bound to carrageenan.

### Example 19

### CARRA/NES (Release Rates)

CARRA/NES

Solutions of increasing concentrations of NES were formulated into the K-II/λ carrageenan composition to establish compatibility of the two compounds. A concentration of 500 μg/mL of NES in the K-II/λ carrageenan composition retained the theological properties, as measured by pH, viscosity, homogeneity and ocular appearance, and exhibited retention of strength, as measured by the HSV-2/mouse assay. This concentration of NES is 40 times higher than the predicted concentration needed for a high-dose formulation of 100 pg/mL.

Diffusion of NES from CARRA/NES was investigated by two different methods, membrane dialysis and Ultrafree-15 centrifugation. In the membrane dialysis experiments, the membrane cutoff is 1,000, and diffusion of NES was measured by HPLC. Results indicate that NES is not bound to the negatively charged carrageenan and, although the rate of diffusion through a dialysis membrane is different than in vivo systemic absorption, diffusion occurs in a time dependent manner. In the Ultrafree-15 centrifugation experiments, a Millipore. Ultrafree-15 centrifugal filter and tube assembly was employed, which allows the passage of molecules of a MW<500 pass through NES MW is 370. The use of this technique demonstrated that 98.6% of NES was recovered.

### Example 20

### Zn/Carrageenan/MIV-150

The combination of the most preferred carrageenans of the present invention along with zinc acetate as the water-soluble metal and MIV-150 as the NNRTI was compared with combinations of zinc/carrageenan (PC-707), MIV-150/carrageenan (PC-815), and carrageenan (Carraguard®). In particular, when animals were challenged with SHIV-RT 8 hours or 24 hours after the last dose of each of these gels, PC-707 and PC-815 separately blocked vaginal SHIV-RT infection at about the same level as Carraguard® (with a p value of greater than 0.05) at the same time at both time points PC-1005 blocked vaginal SHIV-RT infection better than these compounds (with a p value of less than 0.03). Thus, although PC-707 and PC-815 did block vaginal SHIV-RT infection to some extent for up to 24 hours, it was not predictable that the combination of zinc acetate, carrageenans and MIV-150 would totally block vaginal SHIV-RT infection for up to 24 hours.

The experimental procedures were carried out as follows:

A 700 mL glass mixing jar was charged with 1.5 grams of zinc acetate dihydrate, 15.0 grams of the carrageenans (in this case 95% lambda carrageenan and 5% kappa carrageenan), and 300.3 grams of sterile purified water. The contents were mixed with an overhead stirrer at 300 rpm for three hours at ambient temperature. Separately, a 250 ml Erlenmeyer flask was charged with 1.0 grams of methyl paraben and 150.9 grams of sterile purified water. The Erlenmeyer flask was heated to 60° C. with stirring to afford a clear solution, and the contents of the flask were immediately added to the zinc-carrageenan water mixture contained in the 700 ml mixing jar. The mixture was stirred for two hours at ambient temperature at which time 5 mL of an MIV-150/DMSO stock solution prepared by dissolving 18.5 mg of MIV-150 in 10 mL of dimethyl sulfoxide were added to the contents of the 700 mL mixing jar. This mixture was then stirred at 300 rpm for 35 minutes at ambient temperature, and 26 grams of sterile purified water was then added to the mixing jar and the contents were stirred for 30 minutes at ambient temperature.

Macaques were injected with Depo-Provera and three week later given 2 ml of the gel prepared as discussed above per day for two weeks prior to being challenged with 1,000 TClD50 of SHIV-RT at the indicated times after the last gel was applied. The numbers of infected animals as set forth in Table 1 below reflect the number with typical viremia (sly RNA copies in plasma).

### Example 21

A half-sized macaque IVR (20 mm×2 mm) was created using a conventional brass cavity mold having two halves of equal dimensions. A flat brass plate was placed against one half of the mold. Thus, instead of creating a full-sized IVR (20 mm×4 mm) in which both of the cavity molds are joined together and the polymer is injection molded, the flat brass plate provided a half macaque IVR by injection molding. The polymer used was EVA 28, Scientific Polymer Products, which was injection molded at 127° C.

Subsequently, a brass wire 48 mm long (1.62 mm thick) (R. J. Leahy, gauge 14) was Shaped into a ring with an outside diameter of 17.3 mm. It was placed on the flat surface of the half ring and heated at about 110° C. The press wire was embedded in the polymer matrix by gently applying pressure using a fiat brass plate. The embedded brass spring, after heating, was then removed, thus forming a groove in the upper surface of the half ring.

About 80 mg of carrageenan was then manually filled in the groove or channel formed on the flat surface of the half ring. The carrageenan powder was then compressed by placing the same brass wire thereon and using a brass plate, thus packing the carrageenan therein. Maintaining the IVR in the mold, these steps were repeated thus retaining the shape of the IVR and preventing deformation.

Once the carrageenan was filled in the cavity in a compressed form, the mold was joined with the other half of the conventional brass cavity mold; i.e., without any flat brass plate thereon. The polymer then was injected molded to yield a full IVR containing the carrageenan reservoir imbedded therein. A hole was then drilled to permit carrageenan release from the orifice.

The final step of producing the full ring could include a different polymer from that of the step used to produce the half ring. Thus, instead of the EVA 28 used in this Example, the final ring can instead employ any other thermoplastic elastomer that can fuse with the polymer used in the first half of the IVR. The final step in producing a full ring can also include the creation of two halves, both with grooves for containing water-soluble polymer, and/or compounds and then sealing the two halves using a heat seal or bioadhesives. As noted above, the ring itself can be compounded or loaded with various low molecular weight water-insoluble compounds described above.

### Example 22

Another embodiment of the present invention was prepared by first extruding a full-sized macaque EVA ring under normal conditions with a dual mold at 210° C. The ring was produced from the EVA polymer, and preferably is compounded with MIV-150.

Eight holes were then drilled on one surface of the ring throughout the depth of the ring, but not piercing the opposite surface. Carrageenan was then filled into each of these eight holes and manually packed using a metal spatula. In an alternate embodiment, the holes can be filled with a carrageenan-zinc acetate mixture as discussed above. The pellets in each of these eight holes was then compressed with a spatula until no more carrageenan can be added. Each hole contained almost 10 mg of carrageenan, and thus the entire ring contained about 80 mg of carrageenan.

### Example 23

In another embodiment of the present invention, two macaque IVRs (20 mm×4 min) were produced using conventional injection molding, based on EVA polymers. These IVRs were then placed in the mold and the central cavity within the IVRs was filled with nearly 200 mg of carrageenan. The molds were then closed and subjected to three tons of pressure using the Arbor press for nearly one minute. In this manner, a compressed disk of carrageenan was formed within the ring. The compressed carrageenan disk remained whole and sustained its integrity upon removal from the molds. The reservoirs were made with carrageenan alone and with carrageenan-zinc acetate mixtures. With increased amounts of zinc acetate, however, the compression properties were negatively impacted.

Water-impermeable sheets (about 1.2 mm in thickness) were glued to both the anterior and posterior surfaces of these IVRs so that the carrageenan reservoirs were formed within the IVR. The outer layer thus included both the water-impermeable sheets and the surface of the IVR itself. In any event, a hole was drilled through the surface of one of the sheets to act as a releasing pore for the carrageenan.

A separate conventional human-sized IVR was then produced, and these two macaque rings containing the carrageenan cores were then mounted on the human-sized IVR, which itself can also serve as a matrix.

### Example 24

In another embodiment of the present invention, using the molds shown in FIG. 21, a fast IVR half such as that shown in FIG. 15 was produced, in this case using the six-member mold of FIG. 21 and the projections 122 shown in FIG. 21 on the right-hand side to produce the grooves within the IVR halves. The IVR halves were again made from EVA-28 and extruded at around 151° C.

A hydrophilic polyurethane resin identified as PURSIL AL-2075 was loaded with zinc acetate as a low molecular weight water-soluble compound using the solvent casting method. Since the addition of the zinc, acetate lowered the extrusion temperature of the PURSIL AL-2075 significantly, this allowed extrusion of this loaded resin at relatively low temperatures. With each of the IVR halves contained in one of the six molds shown on the left of FIG. 21 an incision was made through the outer wall of the half of the IVR to permit the extruded resin to enter into the groove from the center of the mold. In this manner hot molten resin was enabled to flow into the grooves. To ensure that the resin filled the groove a flat plate was placed upon the top of the left-hand side of the mold shown in FIG. 21. The IVR was then sealed using a normal extrusion process using EVA polymer to create the second half of the ring. As an alternative, a second IVR half including a groove can be produced, and the groove filled either in the same manner as the first half, or with a different core, such as including a high molecular weight water-soluble polymer such as carrageenan or the like. The two halves can then be sealed together to form the IVR.

### REFERENCES

1. Butini L. et al. Intercellular adhesion molecules (ICAM)-1, ICAM-2 and ICAM-3 function as counter receptors for lymphocyte function-associated molecule 1 in human immunodeficiency virus-mediated syncytia formation. Eur J Immunol 24, 2191-2195 (1994).
2. Food and Thug Administration. GRAS (Generally recognized as safe) food ingredients: Carrageenan. FDA Publications PB-221 206, (1972).
3. Benitz K. F., Abraham, R., Golberg, L. & Coulston, F. Carrageenan: an ulcerogenic agent. Toxicol. Appi. Pharmacol. 22, 282 (1972).
4. Fox, MRS. & Jacobs. R. M. Metal Ions in Biological Systems., pp. 214-248 (Marcel Decker. Inc. 1986).
5. Luscombe, D. K. & Nicholls, P. J. Acute and subacute oral toxicity of AHR-2438B, a purified sodium lignosulphonate in rats. Fd. Cosmet. Toxicol. 11, 229-237 (1973).
6. Naess, B. The effect of microbial and animal proteinases on peptide- and protein lignosulphonic acid complexes in agar gel. Acta Vet. Scand. 12, 592-600. 1971. Ref Type: Abstract
7. Samman S. & Roberts, D. C. K. The effect of zinc supplements on plasma zinc and copper levels and the reported symptoms in healthy volunteers. Med. J. Australia 146, 246-249 (1987).
8. U.S.EPA. Health Effects Assessment of for Zinc (and Compounds). EPA/540-1-96-048. 1984. Washington, D.C., US Environmental Protection Agency, Office of Research and Development. Ref Type: Data File
9. Walden, J. T. & Derreth, D. FDA New Release 72/55. FDA Publications 7235, (1972).
10. Walker, A. P. et al. Test guidelines for the assessment of skin tolerance of potentially irritant cosmetic ingredients in man. Fd. Chem. Toxic. 35, 1099-1106 (1997).
11. Weiner, M. L. Intestinal transport of some macromolecules in food. Fd. Chem. Toxic. 26, 10, 867-880 (1988).
12. Food and Drug Administration. Study of mutagenic effects of calcium carrageenan (FDA No. 71-5). FDA Publications PB-221 820, (1972).
13. Elias, C. J. et al. Colposcopic Evaluation of a Vaginal Gel Formulation of iota-Carrageenan. Contraception 56, 387-389 (1997).
14. Lines, A. D. Value of the K+ Salt of Carrageenan as an Agar Substitute in Routine Bacteriological Media. Applied and Environmental Microbiology 34, 637-639 (1977).
15. Phillips, D. M. & Tan, X. Mechanism of trophoblast infection by HIV. AIDS Res Hum Retroviruses 9, 1697-1705 (1992).
16. Baba, M. et al. Pentosan polysulfate, a sulfated oligosaccharide, is a potent and selective anti-HIV agent in vitro. Antiviral Res 9, 335-343 (1988).
17. Baba, M. et al. Mechanism of inhibitory effect of dextran sulfate and heparin on replication of human immunodeficiency virus in vitro. Proc Natl Acad Sci 85, 6132-6126 (1988).
18. Pearce-Pratt, R. & Phillips. D. M. Studies of adhesion of lymphocytic cells: Implications for sexual transmission of human immunodeficiency virus. Biol. Reprod 48, 431-445 (1993).
19. Pearce-Pratt, R. & Phillips, D. M. Sulfated polysaccharides lymphocyte-to-epithelial transmission of HIV-1. Biol. Reprod 54, 173-182 (1996).
20. Maguire, R. A., Zacharopoulos, V. R. & Phillips, D. M. Carrageenan-N9 Spermicides for Preventing Pregnancy and Sexually Transmitted Infections. Sex Transm Dis 25, 494-500 (1998).
21. Phillips, D. M. Perspectives in Drug Discovery and Design. Fantini, S. & Sabatier, J. M. (eds.), pp. 213-223 (1996).
22. Zacharopoulos, V. R. & Phillips. D. M. Vaginal formulations of carrageenan protect mice from herpes simplex virus infection. clin. Diag. Lab. Immunol. 4, 465-468 (1997).
23. Phillips, D. M. & Zacharopoulos, V. R. Nonoxynol-9 Enhances Rectal Infection by Herpes Simplex Virus in Mice. Contraception 57, 341-348 (1998).
24. Masuner, C. et al. Dendritic cells route Human Immunodficiency Virus to lymph nodes after vaginal or intravenous administration to mice. J Virol 72, 7822-7829 (1998).
25. Masuner, C. et al. Dendritic Cells in Fundamental and Clinical Immunology. Riccciardi-Castagnoli (ed.), pp. 411-414 (Plenum Press, New York, 1997).
26. Anderson, D. J. Mechanisms of HIV-1 transmission via semen. J. NIH Res. 4, 104-111 (1992).
27. Levy, J. A. The transmission of AIDS: the case of the infected cell. JAMA 259, 3037-3038 (1988).
28. Zacharopoulos, V. R., Perotti, M. E. & Phillips. D. M. A role for cell migration in the sexual transmission of HIV? Current Biol. 7, 534-537 (1997).
29. Howett M. K., K.J.W.C.K.D. Human xenogafts. A model system for human papillomavirus infection. Intervir. 31, 109-115 (1990).
30. Jerse, A. E. Experimental gonococcal genital tract infection and opacity protein expression in estradio-treat mice. Infect Immun 67, 5699-5708 (1999).
31. Kolopp. M. et al. Predictive value of an in vitro model for skin initation (SkinEthic) applied to the testing of topical vehicles for SDZ ASM 981. Proc. Clin. Dermatol. 2000 Singapore 141. 1998. Ref Type: Abstract
32. Mitsuya, H. et al. Dextran sulfate suppression of viruses in the HTV family: inhibition of virion binding to CD4+ cells. Science 240, 646-649 (1988).
33. Suzuki H, T.T.I.K.Y.S.Y.N.T.S. Lignosulfonate a water-solubilized lignin from the waste liquor of the pulping process, inhibits the infectivity and cytopathic effects of Human Immunodeficiency Virus in Vitro. Agric Bid Chem 53, 3369-3372 (1989).
34. Baba, M., Schols, D., Pauwels R., Nakashima, H. & De Clercq, E. Sulfated polysaccharides as potent inhibitors of HIV-induced syncytium formation: a new strategy towards ADS chemotherapy JAIDS 3, 493-499 (1990).
35. NRC (National Research Council). Recommended Dietary Allowances. (National Academy Press, Washington D.C., 1989).
36. Nicklin. S. & Miller, K. Effect of orally administered food-grade carrageenans on antibody-mediated and cell-mediated immunity in the inbred rat. Fd. Chem. Toxic. 228, 615-621 (1984).
37. CEAMSA (Compania Espanola de Algas Mammas, S. A. Technical Information 1999-2000. 1999. South America. Ref Type: Data File
38. ATSDR (Agency for Toxic Substances and Disease Registry). Toxological Profile for Zinc. Agency for Toxic Substances and Disease Registry, U.S. Public Health Service, Atlanta, Ga. 121 pp (2001).
39. Haraguchi, Y., Sakurai, H., Hussain, S., Anner, B. & Hoshino, H. Inhibition of HIV-1 Infection by Zinc Group Metal Compounds. Antiviral Res 43, 123-133 (1999).
40. Sergio, W. Zinc Salts that may be Effective Against the ADS Virus HIV. Medical Hypotheses 261, 253 (1988).
41. Arens, M. & Travis, S. Zinc Salts Inactivate Clinical Isolates of Herpes Simplex Virus In Vitro. J Clin Microbiol 38, 1758-1762 (2000).
42. Tennican, P., Carl, G., Frey, J., Thies, C. & Chvapil, M. Topical Zinc in the Treatment of Mice Infected Intravaginally with Herpes Genitalis Virus. Proceedings of the Society tbr Experimental Biology and Medicine 164, 593-597 (1980).
43. Alvarez-Sanchez, F., Brache, V. Jackanicz, T. & Faundes, A. Evaluation of four different contraceptive vaginal rings: steroid serum levels, luteal activity, bleeding control and lipid profiles. Contraception 46, 387-397 (1992).
44. Mishell, D. J., Lumkin, M. & Jackanicz, T. Initial clinical studies of intravaginal rings containing norethindrone and norgestrel. Contraception 12, 253 (1975).
45. Ballagh, S., Mishell, D., Jackanicz, T., Lacarra, M. & Eggena, P. Dose-finding study of a contraceptive ring releasing norethindrone acetate/ethinyl-estradiol. Contraception 50, 535-549 (1994).
46. Sivin, I. Mishell, D. J., Victor, A. & et al. A multicenter study of levonorgestrelestradiol contraceptive vaginal rings II—use of effectiveness. Contraception 24, 341-358 (1981).
47. Sivin, I., Mishell, D. J., Victor, A. & et al. A multicenter study of levonorgestrelestradiol contraceptive vaginal rings II—subjective and objective measures of effects. An international comparative trial. Contraception 24, 359-376 (2001).
48. Fanchin R et al. Transvaginal administration of progesterone: dose-response data support a first uterine pass effect. Obstet Gynecol 90, 396-40 1 (1997).
49. Cicinelli, E., Cignarelli, M., Sabatelli, S. & et al. Plasma concentrations of progesterone are higher in the uterine artery than in the radial artery after vaginal administration of hicronized progesterone in an oil-based solution to postmenopausal women. Fertil Steril 69, 471-473 (1998).
50. Rigg, L., Milanes, B., Villanueva &. Yen, S. Efficacy of intravaginal and intranasal administration of micronized estradiol-17B. JCE&M 45, 1261-1264 (1977).
51. Martin, P. et al. Estradiol, estrone, and gonadotropin levels after use of vaginal estradiol. Obstet & Gyn 63, 441-444 (1984).
52. Schiff, I., Tulchinsky, D. & Yan, K. Vaginal absorption of esterone and 17B-estradiol. Fertil Steril 28, 1063-1066 (1977).
53. Kumar, N., Koide, S., Tsong Y & Sundaram, K. Nestorone: a progestin with a unique pharmacologic profile. Steroid 65, 629-636 (2000).
54. Odlind, V., Lithell, H., Selinus, I. & Vessby, B. Unaltered lipoprotein and carbohydrate metabolism during treatment with contraceptive subdermal implants containing 5T1435. Contraception 31, 130 (1985).
55. Robins, A. & Bardin, C. Nestorone Progestin—the ideal progestin for use in controlled release delivery systems. Ann N Y Acad Sci 828, 38-46 (1997).
56. Massai, R., Diaz 5, Jackanicz. I. & Croxatto R B. Vaginal rings for contraception in lactating women. Steroid 65, 703-707 (2000),
57. Lahteenmaki P L A, Daz 5, Miranda P & Croxatto K B. Milk and plasma concentrations of the progestin ST 1435 in women treated parenterally with ST 1435. Contraception 42, 555-562 (1990).
58. World Health Organization. Microdose intravaginal levonorgestrel contraception: a multicentered clinical trial I. Contraception 41, 105-124 (1990).
59. Yen, S. Reproductive Endocrinology. Yen S S C and Jaffe R B (ed.), pp. 200-236 (W.B. Saunders Co, Philadelphia. 1986).
60. Henzl, M. Reproductive Endocrinology. Yen S S C and Jaffe R B (ed.), pp. 243-682 (W.B. Saunders Co., Philadelphia, 1986).
61. Brache V. et al. Ovarian function during use of vaginal rings delivering three different doses of Nestorone. Contraception. 2001. Ref Type: In Press
62. Fraser I et al. Vaginal epithelial surface appearances in women using vaginal rings for contraception. Contraception 61, 13 1-138 (2000).
63. Couch, R. C. A 12-month systemic toxicity study of subdermal implant for 5T1435 in female cynomolous monkeys. New Mexico Regional Primate Research Laboratory, New Mexico State University Holloman AFB New Mexico. Population Council Files. 1992. Ref Type: Unpublished Work
64. Lahteenmaki, P., Weiner, E., Johansson, E. & Luukkainen. T. Contraception with subcutaneous capsules containing 5T1435. Pituitary and ovarian function and plasma levels of 5t1435. Contraception 23, 63-75 (1981).
65. Lahteenmaki P. Weiner E. Johansson E &. Luukkainen T. Pituitary and ovarian function during contraception with one subcutaneous implant releasing a progestin. 5T1435. Contraception 25, 299-306 (1982).
66. Haukkamaa, M., Laurikka-Routti, M. & Heikinheimo, O. Transdermal absorption of the progestin 5T1435: Therapeutic serum steroid concentrations and high excretion of the steroid in saliva. Contraception 44, 269-276 (1991).
67. Laurikka-Routti M., Haukkamaa, M. & Lahteenmaki, P. Suppression of ovarian function with the transdermally given synthetic progestin 5T1435. Fertil Steril 58, 680-684 (1992).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A vaginal ring for the delivery of a water-soluble polymer comprising an outer layer of a non-water-swellable elastomer, an inner layer of said water-soluble compound, and at least one aperture in said outer layer to permit release of said water-soluble compound from said inner layer thereof only through said at least one aperture.

2. The vaginal ring of claim 1 wherein said water-soluble compound comprises a high molecular weight water-soluble polymer.

3. The vaginal ring of claim 1 wherein said outer layer comprises an extruded polymeric ring, and said inner layer is encased within said extruded polymeric ring, whereby said outer layer comprises the only layer encasing said inner layer.

4. The vaginal ring of claim 1 wherein said non-water-swellable elastomer is capable of controllably diffusing a water-insoluble compound therethrough.

5. The vaginal ring of claim 2 wherein said high molecular weight water-soluble polymer is free of any polymeric coating or layer which will prevent the release of said high molecular weight water-soluble polymer therefrom.

6. The vaginal ring of claim 1 wherein said outer layer comprises an extruded polymeric ring and including a polymeric sheath surrounding said inner layer, said at least one aperture being provided in both said outer layer and said polymeric sheath.

7. The vaginal ring of claim 6, wherein said polymeric sheath includes an active agent selected from the group consisting of water soluble and water insoluble compounds.

8. The vaginal ring of claim 6 wherein said polymeric, sheath comprises a non-water-swellable elastomer.

9. The vaginal ring of claim 8 wherein said non-water-swellable elastomer of said polymeric sheath comprises the same non-water-swellable elastomer of said outer layer.

10. The vaginal ring of claim 3 wherein said extruded polymeric ring comprises a polymer selected from the group consisting of EVA and silicone polymers.

11. The vaginal ring of claim 2 wherein said inner layer comprises a compressed core of said high molecular weight water-soluble polymer.

12. The vaginal ring of claim 11 wherein said high molecular weight water-soluble polymer is selected from the group consisting of carrageenan, proteins, polylactic acids, peptide hormones, enzymes, and cell adhesion molecules.

13. The vaginal ring of claim 12 wherein said high molecular weight water-soluble polymer comprises carrageenan.

14. The vaginal ring of claim 1 wherein said outer layer includes at least one water-insoluble compound.

15. The vaginal ring of claim 14 wherein said at least one water-insoluble compound is compounded with said non-water-swellable elastomer.

16. The vaginal ring of claim 15 wherein said at least one water-insoluble compound is selected from the group consisting of NNRTIs.

17. The vaginal ring of claim 16 wherein said at least one water-insoluble compound comprises MIV-150.

18. The vaginal ring of claim 1 wherein said water-swellable polymer comprises a low molecular weight water-soluble compound.

19. The vaginal ring of claim 18 wherein said low molecular weight water-soluble compound comprises a zinc salt.

20. The vaginal ring of claim 19 wherein said low molecular weight water-soluble compound is admixed with an elastomeric polymer which enables the sustained release of said low molecular weight water-soluble compound therefrom.

21. The vaginal ring of claim 3 wherein said inner layer comprises a pair of said inner layers.

22. The vaginal ring of claim 21 wherein said pair of inner layers includes a first inner layer comprising a compressed core of a high molecular weight water-soluble polymer and a second inner layer comprising a compressed core of a low molecular weight water-soluble compound.

23. The vaginal ring of claim 22 wherein said inner layer includes an elastomeric polymer which enables the sustained release of said low molecular weight water-soluble compound therefrom.

24. The vaginal ring of claim 23 wherein said elastomeric polymer comprises a hydrophilic or hydrophobic elastomeric polymer.

25. The vaginal ring of claim 2 wherein said outer layer is in the form of a ring, said ring including said at least one aperture extending substantially transversely through at least a portion of said ring, and wherein said inner layer comprises a compressed pellet of said high molecular weight water-soluble polymer disposed in said at least one aperture.

26. The vaginal ring of claim 25 wherein said ring includes a plurality of said apertures, and said inner layer comprises a plurality of said compressed pellets of said high molecular weight water-soluble polymer disposed in said plurality of apertures.

27. The vaginal ring of claim 25 wherein said ring includes a plurality of said apertures, and said inner layer comprises at least one compressed pellet of said high molecular weight water-soluble polymer disposed in at least one of said plurality of apertures and at least one pellet of a low molecular weight water-soluble compound disposed in at least one other of said plurality of apertures.

28. The vaginal ring of claim 27 wherein said at least one pellet of a low molecular weight water-soluble compound includes an elastomeric polymer which enables the sustained release of said low molecular weight water-soluble compound therefrom.

29. The vaginal ring of claim 28 wherein said elastomeric polymer comprises a hydrophilic or hydrophobic elastomeric polymer.

30. The vaginal ring of claim 25 wherein said ring includes at least one low molecular weight water-insoluble compound.

31. The vaginal ring of claim 30 wherein said at least one water-insoluble compound is selected from the group consisting of NNRTIs.

32. The vaginal ring of claim 31 wherein said at least one water-insoluble compound comprises MIV-150.

33. The vaginal ring of claim 30 wherein said at least one low molecular weight water-insoluble compound is compounded with said non-water-swellable elastomer comprising said ring.

34. The vaginal ring of claim 25 wherein said compressed core includes at least one low molecular weight water-soluble compound.

35. The vaginal ring of claim 34 wherein said low molecular weight water-soluble compound comprises a zinc salt.

36. The vaginal ring of claim 28 wherein said low molecular weight water-soluble compound comprises a zinc salt.

37. The vaginal ring of claim 28 wherein said high molecular weight water-soluble polymer comprises carrageenan.

38. The vaginal ring of claim 2 wherein said outer layer comprises at least one ring of said water-impermeable polymeric compound, and said inner layer comprises a compressed core of said high molecular weight water-soluble polymer contained within the center of said ring.

39. The vaginal ring of claim 38 wherein said ring includes an upper surface and a lower surface, and wherein said outer layer includes a pair of outer sheets of non-water-swellable elastomers disposed on said upper and lower surfaces of said ring, said at least one aperture extending through at least one of said pair of outer sheets.

40. The vaginal ring of claim 39 wherein said non-water-swellable elastomers of said ring and said non-water-swellable elastomers of said pair of sheets comprise the same non-water-swellable elastomers.

41. The vaginal ring of claim 40 wherein said non-water-swellable elastomers comprise EVA or a silicone polymer.

42. The vaginal ring of claim 2 wherein said outer layer comprises a plurality of rings of said non-water-swellable elastomers and said inner layer comprises a plurality of compressed cores of said high molecular weight water-soluble polymer contained within the centers of each of said plurality of rings.

43. The vaginal ring of claim 39 wherein said outer layer comprises a plurality of rings of said non-water-swellable elastomers and said inner layer comprises a plurality of compressed cores of said high molecular weight water-soluble polymer contained within the centers of each of said plurality of rings.

44. The vaginal ring of claim 39 wherein said outer layer comprises a plurality of rings of said non-water-swellable elastomers and said inner layer comprises at least one inner layer comprising a compressed core of said high molecular weight water-soluble polymer disposed in at least one of said plurality of rings and at least one other inner layer of a compressed core of a low molecular weight water-soluble compound disposed in at least one other of said plurality of rings.

45. The vaginal ring of claim 44 wherein said compressed core of said low molecular weight water-soluble compound includes an elastomeric polymer which enables the sustained release of said low molecular weight water-soluble compound therefrom.

46. The vaginal ring of claim 44 wherein said elastomeric polymer comprises a hydrophilic or hydrophobic elastomeric polymer.

47. The vaginal ring of claim 38 including a separate ring encompassing said at least one ring.

48. The vaginal ring of claim 47 wherein said separate ring comprises a polymer selected from the group consisting of thermoplastic and thermosetting elastomers, and including a low molecular weight water-insoluble compound.

49. The vaginal ring of claim 48 wherein said low molecular weight water-insoluble compound is compounded with said polymer selected from the group consisting of thermoplastic and thermosetting elastomers comprising said separate ring.

50. The vaginal ring of claim 42 including a separate ring encompassing said plurality of rings.

51. The vaginal ring of claim 49 wherein said separate ring comprises a polymer selected from the group consisting of thermoplastic and thermosetting elastomers, and including a low molecular weight water-insoluble compound.

52. The vaginal ring of claim 51 wherein said low molecular weight water-insoluble compound is compounded with said polymer selected from the group consisting of thermoplastic and thermosetting elastomers comprising said separate ring.

53. The vaginal ring of claim 45 including a separate ring encompassing said at least one ring.

54. The vaginal ring of claim 53 wherein said separate ring comprises a polymer selected from the group consisting of thermoplastic and thermosetting elastomers, and including a low molecular weight water-insoluble compound.

55. The vaginal ring of claim 54 wherein said low molecular weight water-insoluble compound is compounded with said polymer selected from the group consisting of thermoplastic and thermosetting elastomers comprising said separate ring.

56. A method of manufacturing a vaginal ring for delivery of a water-soluble polymer comprising preparing said outer layer of a non-water-swellable elastomer, preparing an inner layer of said water-soluble compound, disposing said inner layer within said outer layer, and providing at least one aperture in said outer layer whereby said water-soluble compound can only be delivered through said at least one aperture.

57. The method of claim 56 wherein said water-soluble compound comprises a high molecular weight water-swellable polymer.

58. The method of claim 56 wherein said preparing of said outer layer comprises forming said outer layer in the form of a ring.

59. The method of claim 58 wherein said preparing said inner layer of said water-soluble polymer comprises leaving the outer surface of said inner layer free of any polymeric coating or layer which would prevent the release of said high molecular weight water-soluble polymer therefrom.

60. The method of claim 58 wherein said preparing of said inner layer comprises surrounding said inner layer with a polymeric sheath and wherein providing of said at least one aperture in said outer layer includes providing said at least one aperture in said polymeric sheath.

61. The method of claim 60 including combining an active agent selected from the group of water soluble and water insoluble compounds with said polymeric sheath.

62. The method of claim 60 including combining said non-water-swellable elastomer with at least one low molecular weight water insoluble compound.

63. The method of claim 58 wherein said preparing of said outer layer comprises forming a first portion of said ring including an inner surface and an outer surface, and preparing a second portion of said ring including an inner surface and an outer surface, and combining said first and second portions of said ring by juxtaposing said inner surfaces of said first and second portions to fully form said ring.

64. The method of claim 63 including providing at least one groove on said inner surface of one of said first and second portions of said ring.

65. The method of claim 64 including disposing said high molecular weight water soluble polymer in said at least one groove prior to combining said first and second portions of said ring.

66. The method of claim 63 including providing at least one groove on the inner surfaces of each of said first and second portions of said ring.

67. The method of claim 66 including disposing said high molecular weight water-soluble polymer in one of said at least two grooves, and disposing a low molecular weight water-soluble compound in the other of said at least two grooves.

68. The method of claim 63 including providing said at least one aperture transversely through said ring.

69. The method of claim 68 wherein said at least one aperture substantially traverses the depth of said ring.

70. The method of claim 68 wherein said disposing of said inner layer within said outer layer comprises inserting a compressed pellet of said inner layer within said at least one aperture.

71. The method of claim 63 including providing a plurality of said apertures transversely through in said ring.

72. The method of claim 71 wherein said plurality of apertures substantially transverse the depth of said ring.

73. The method of claim 72 wherein said disposing of said inner layer within said outer layer comprises inserting said high molecular weight water-soluble polymer within said plurality of apertures.

74. The method of claim 68 including combining said non-water-swellable elastomer with a low molecular weight water-insoluble compound.

75. The method of claim 73 including combining said high molecular weight water-soluble polymer with a low molecular weight water-soluble compound.

76. The method of claim 75 wherein said preparation of said inner layer comprises compressing said high molecular weight water-soluble polymer.

77. The method of claim 71 wherein said disposing of said inner layer within said outer layer comprises inserting said high molecular weight water-soluble polymer in at least one of said plurality of apertures, and including inserting said low molecular weight water-soluble compound in at least one other of said plurality of apertures.

78. The method of claim 77 wherein said high molecular weight water-soluble polymer comprises carrageenan and said low molecular weight water-soluble compound comprises a zinc salt.

79. The method of claim 56 wherein said disposing of said inner layer within said outer layer comprises providing said compressed water-soluble polymer within the center of said ring.

80. The method of claim 79 wherein said ring includes an upper surface and a lower surface, and wherein said providing said outer layer comprises providing a first sheet of non-water-swellable elastomer on said upper surface of said ring and providing a second sheet of non-water-swellable elastomer on said lower surface of said ring, and wherein said at least one aperture is disposed in one of said first and second sheets.

81. The method of claim 57 wherein said preparation of said outer layer comprises forming a plurality of outer layers in the form of a plurality of rings.

82. The method of claim 58 wherein said water-soluble polymer comprises a high molecular weight water-soluble polymer, and said preparation of said inner layer comprises compressing a plurality of cores of said high molecular weight water-soluble polymer.

83. The method of claim 82 wherein said disposing of said inner layers within said outer layers comprises providing said plurality of compressed cores of said high molecular weight water-soluble polymer within the centers of said plurality of rings.

84. The method of claim 83 wherein said plurality of rings include upper surfaces and lower surfaces, and wherein said providing said outer layer includes providing a plurality of first sheets of non-water-swellable elastomer on said upper surfaces of said plurality of rings and providing a plurality of second sheets of non-water-swellable elastomers onto said lower surfaces of said plurality of rings, and disposing a plurality of said at least one apertures in one of said first and second pluralities of sheets.

85. The method of claim 84 including preparing a separate ring larger than said plurality of rings and encompassing said plurality of rings within said separate ring.

86. The method of claim 60 wherein said separate ring comprises a polymer selected from the group consisting of thermoplastic and thermosetting elastomers.

87. The method of claim 86 including combining a low molecular weight water-insoluble compound with said polymer.

88. The method of claim 85 wherein said preparation of said inner layer comprises compressing at least one core of said high molecular weight water-soluble polymer and including disposing said at least one core of said high molecular weight water-soluble polymer in at least one of said plurality of rings, and including providing at least one core of a low molecular weight water-soluble compound and disposing said at least one core of said low molecular weight water-soluble compound in another of said plurality of rings.
